(19) United States
(12) Patent Application Publication
(10) Pub. No.: US 2023/0263768 A1
(43) Pub. Date: Aug. 24, 2023

(54) WATER-SOLUBLE ARTESUNATE-BASED THERAPY FOR CORONAVIRUS INFECTION

(71) Applicant: SOLSTAR PHARMA, Laval (CA)

(72) Inventors: Tien Canh LE, Montreal (CA); Dionissios BALTZIS, Laval (CA)

(21) Appl. No.: 18/012,536

(22) PCT Filed: Jun. 23, 2021
(86) PCT No.: PCT/CA2021/050864

§ 371 Date: Dec. 22, 2022

Related U.S. Application Data
(60) Provisional application No. 63/044,384, filed on Jun. 26, 2020.

(51) Int. Cl. A61K 31/357; A61K 33/30; A61K 9/20; A61K 31/4184; A61P 31/14; A61K 31/573; A61K 9/00; A61K 31/245; A61K 31/167; A61K 31/4045; A61K 31/15; A61K 31/593

(52) U.S. Cl. CPC A61K 31/357; A61K 9/0053; A61K 9/2013; A61K 9/2027; A61K 9/2054; A61K 31/15; A61K 31/167; A61K 31/245; A61K 31/4045; A61K 31/4184; A61K 31/573; A61K 31/593; A61K 33/30; A61P 31/14

(57) ABSTRACT

The present document describes methods of treating or preventing a viral infection in a subject in need thereof comprising administering a therapeutically effective amount of artesunate or pharmaceutically acceptable salts thereof, and stereoisomers thereof to said subject. More particularly, the present document describes methods of treating or preventing a viral infection in a subject in need thereof comprising administering a therapeutically effective amount of a delayed release dosage form comprising an artesunate or pharmaceutically acceptable salts thereof, in combination with a carbonate salt, an artesunate emulsion having a pH value of from about 7.5 to 8.0 and comprising an artesunate or pharmaceutically acceptable salts thereof, and stereoisomers thereof stabilized with an emulsifying polymer and a soluble polymers, or a combination thereof, to the subject.

### CROSS-REFERENCE TO RELATED APPLICATIONS

This application claims priority of U.S. provisional Pat. application No. 63/044,384 filed on Jun. 26, 2020, the specification of which is hereby incorporated by reference in its entirety.

### BACKGROUND

### (A) Field

The subject matter disclosed generally relates to delayed release dosage forms. More specifically, the subject matter disclosed relates to the use of delayed release dosage forms comprising artesunate for the treatment of corona virus infection.

### (B) Related Prior Art

Recently, a novel Coronavirus Disease 2019 (COVID-19) responsible for Severe Acute Respiratory Syndrome (SARS-CoV-2) has spread globally as a serious pandemic. For the moment, no validated therapeutics against virus-target interactions are available for COVID-19 and a rapid and efficient treatment for SARS-CoV-2 is urgently needed.

The novel coronavirus pneumonia (COVID-19) is a contagious acute respiratory infectious disease. After several tests and nucleic acid sequencing, researchers confirmed that the nucleic acid of the novel coronavirus is a positive-stranded RNA. Its structural proteins include: Spike Protein (S), envelope protein (E), membrane protein (M), and nucleocapsid phosphoprotein (See FIG. 1). Generally, patients with the coronavirus pneumonia show fever with temperature superior to 37.5° C., with symptoms such as dry cough, fatigue, difficulty breathing, and frost-glass-like symptoms in the lung. The disease is highly transmitted, and the number of infected people has reached millions around the world. Most mild patients get a better prognosis. Some serious patients quickly develop acute respiratory distress syndrome, shock, acidosis, coagulopathy, and even death. For this reason, there is an urgent need for effective treatment. Current focus has been on the development of novel therapeutics, including antivirals and vaccines.

Severe acute respiratory syndrome (SARS) and the cytokine storm account for a large part of the high mortality in intensive care units.

Artesunate is a hemi-succinate derivative of artemisinin. Artesunate is unstable in aqueous, acidic and basic conditions, and is sensitive to light. Also, salt forms of artesunate (such as sodium artesunate) are sticking, have low flowability, and are difficult to handle. Artesunate possesses anti-inflammatory properties, via its attenuation of the production of interleukins (IL)-1β, IL-6 and IL-8 in tumor necrosis factor (TNF)-α-signaling pathway via the regulation of NF-κB and phosphoinositide 3 kinase (Pl3K) pathways. Artemisinin and its derivatives can regulate the expression of pro-inflammatory cytokines, nuclear factor-kappa B (NF-κB), matrix metalloproteinases (MMPs), and vascular endothelial growth factor (VEGF), promote cell cycle arrest and drive reactive oxygen species (ROS) production.

In this urgent case, it is preferable to formulate a combination therapy using existing approved drugs with proven safety profiles to address the immediate need to reduce the rising mortality. In the present application, artesunate may be used because it is known to be a safe, low cost drug which has been used by thousands of patients with malaria without important adverse effects. Artesunate is recommended by the World Health Organization (WHO) as the treatment of choice for severe malaria. Although the mechanism behind the antimalarial activity of artesunate is not entirely clear, it is thought that the active metabolite, Dihydroartemisinin (DHA) is responsible for the death of the parasite.

The present invention proposes the use of a water-soluble form of artesunate for the treatment or prevention of corona virus disease (COVID).

### SUMMARY

According to an embodiment, there is provided a method of treating or preventing a viral infection in a subject in need thereof comprising administering a therapeutically effective amount of artesunate or pharmaceutically acceptable salts thereof, and stereoisomers thereof to the subject.

According to another embodiment, there is provided a method of treating or preventing a viral infection in a subject in need thereof comprising administering
a therapeutically effective amount of a delayed release dosage form comprising:
   an artesunate or pharmaceutically acceptable salts thereof, in combination with a carbonate salt;
   an artesunate emulsion having a pH value of from about 7.5 to 8.0 and comprising an artesunate or pharmaceutically acceptable salts thereof, and stereoisomers thereof stabilized with an emulsifying polymer and a soluble polymer,
   or a combination thereof,
to the subject.

The artesunate emulsion may comprise from about 25% to about 90% w/w of the artesunate pharmaceutically acceptable salt.

The artesunate emulsion may comprise from about 80% to about 90% w/w of the artesunate pharmaceutically acceptable salt.

The artesunate emulsion may comprises from about 1% to about 55% w/w of the emulsifying polymer.

The artesunate emulsion may comprise from about 5% to about 10% w/w of the emulsifying polymer.

The artesunate emulsion may comprise from about 1% to about 55% w/w of the soluble polymer.

The artesunate emulsion may comprise from about 5% to about 10% w/w of the soluble polymer.

The emulsifying polymer may be a nonionic, an anionic, a cationic, an amphoteric polymer, or a combination thereof.

The emulsifying polymer may be selected from the group consisting of a (hydroxypropyl)methyl cellulose, a methyl cellulose, an ethyl cellulose, an acetyl cellulose, an octenyl succinate starch, a hydroxypropyl starch, a polyvinyl pyrrolidone, a polyvinyl acetate-acrylate, a poloxamer, an albumin, a gelatin or combinations thereof.

The emulsifying polymer may be polyvinyl pyrrolidone.

The soluble polymer may be a nonionic, an anionic, a cationic, an amphoteric polymer, or a combination thereof.

The soluble polymer may be selected from the group consisting of a carboxymethyl cellulose, a carboxymethyl starch, a carboxyethyl starch, a succinyl starch, a distarch glycerol, a distarch phosphate, a hydroxypropyl distarch glycerol, a hydroxypropyl distarch phosphate, a maltodextrin, a cyclodextrin, an acacia gum, a pectin, an amylopectin, a carrageenan, a xanthan gum, a tragacanth gum, a guar gum or combination thereof.

The soluble polymer may be carboxymethyl cellulose.

The pH value may be obtained with a weak base.

The weak base may be a carbonate salt.

The carbonate salt may be selected from the group consisting of sodium carbonate (Na₂CO₃), potassium carbonate (K₂CO₃), sodium bicarbonate (NaHCO₃), and potassium and bicarbonate (KHCO₃).

The carbonate salt may be from about 1% to about 40% w/w of the delayed release dosage form or the artesunate emulsion.

The carbonate salt may be from about 5% w/w of the delayed release dosage form or the artesunate emulsion.

The method may further comprise administering a second therapeutic agent.

The second therapeutic agent may be an antiviral agent, an anti-inflammatory drug, an immunomodulator, an Angiotensin Converting Enzyme (ACE) inhibitor, a chemotherapeutic agent and combinations thereof.

The antiviral agent may be Favipiravir, Abacavir, Acyclovir, Adefovir, Amantadine, Ampligen, Amprenavir, Arbidol, Atazanavir, Atripla, Balavir, Baloxavir marboxil, Biktarvy, Boceprevir, Cidofovir, Cobicistat, Combivir, Daclatasvir, Darunavir, Delavirdine, Descovy, Didanosine, Docosanol, Dolutegravir, Doravirine, Ecoliever, Edoxudine, Efavirenz, Elvitegravir, Emtricitabine, Enfuvirtide, Entecavir, Etravirine, Famciclovir, Fomivirsen, Fosamprenavir, Foscarnet, Fosfonet, Ganciclovir, Ibacitabine, Ibalizumab, Idoxuridine, Imiquimod, Imunovir, Indinavir, Inosine, an Integrase inhibitor, Interferon type I, Interferon type II, Interferon type III, Interferon, Lamivudine, Letermovir, Lopinavir, Loviride, Maraviroc, Methisazone, Moroxydine, Nelfinavir, Nevirapine, Nexavir, Nitazoxanide, Norvir, a nucleoside analogues, Oseltamivir, Peginterferon alfa-2a, Peginterferon alfa-2b, Penciclovir, Peramivir, Pleconaril, Podophyllotoxin, a protease inhibitor, Pyramidine, Raltegravir, Remdesivir, a reverse transcriptase inhibitor, Ribavirin, Rilpivirine, Rimantadine, Ritonavir, Saquinavir, Simeprevir, Sofosbuvir, Stavudine, Telaprevir, Telbivudine, Tenofovir alafenamide, Tenofovir disoproxil, Tenofovir, Tipranavir, Trifluridine, Trizivir, Tromantadine, Truvada, Umifenovir, Valaciclovir, Valganciclovir, Vicriviroc, Vidarabine, Viramidine, Zalcitabine, Zanamivir, Zidovudine, Tilorone, Mepacrine, pyronaridine and combinations thereof.

The anti-inflammatory drug may be a Non-Steroidal Anti-Inflammatory Drugs (NTHEs) comprising aspirin,, celecoxib, diclofenac, diflunisal, etodolac, ibuprofen, indomethacin, ketoprofen, ketorolac, nabumetone, naproxen, oxaprozin, piroxicam, salsalate, sulindac, tolmetin, indomethacin, including colchicine, and analgesic such as acetaminophen and combinations thereof.

The anti-inflammatory drug may be a corticosteroidal drug comprising Dexamethasone, Betamethasone, Prednisone, Prednisolone, Methylprednisolone, Triamcinolone, Budesonide, Flunisolide and combinations thereof.

The immunomodulator may be Anakinra, Canakinumab, Tocilizumab, Sarilumab, Baricitinib, Fedratinib, Ruxolitinib, Fingolimob, Infliximab, Adalimumab, and combinations thereof.

The ACE inhibitor may be Benazepril, Captopril, Enalapril, Fosinopril, Lisinopril, Moexipril, Perindopril, Quinapril, Ramipril, Trandolapril, and combinations thereof.

The ACE blocker may be Telmisartan, Candesartan, Irbesartan, Valsartan, Losartan, Olmesartan, Eprosartan, Azilsartan and combinations thereof.

Transmembrane Protease Serine 2 (TMPRSS2) inhibitor may be Camostat, Nafamostat, Gabexate and combination thereof.

The chemotherapeutic agent may be Daunorubicin, Mitoxantrone, Metamizole and combinations thereof.

The viral infection may be a corona virus infection.

The corona virus infection may be a SARS-CoV-2 (or its variants) infection.

According to another embodiment, there is provided a pharmaceutical composition comprising artesunate or pharmaceutically acceptable salts thereof, for use for the prevention or treatment of a viral infection.

According to another embodiment, there is provided a delayed release dosage form comprising
an artesunate or pharmaceutically acceptable salts thereof, in combination with a carbonate salt;
an artesunate emulsion having a pH value of from about 7.5 to 8.0 and comprising an artesunate or pharmaceutically acceptable salts thereof, and stereoisomers thereof stabilized with an emulsifying polymer and a soluble polymer;
or a combination thereof.

The artesunate emulsion may comprise from about 25% to about 90% w/w of the artesunate pharmaceutically acceptable salt.

The artesunate emulsion may comprise from about 80% to about 90% w/w of the artesunate pharmaceutically acceptable salt.

The artesunate emulsion may comprises from about 1% to about 55% w/w of the emulsifying polymer.

The artesunate emulsion may comprise from about 5% to about 10% w/w of the emulsifying polymer.

The artesunate emulsion may comprise from about 1% to about 55% w/w of the soluble polymer.

The artesunate emulsion may comprise from about 5% to about 10% w/w of the soluble polymer.

The emulsifying polymer may be a nonionic, an anionic, a cationic, an amphoteric polymer, or a combination thereof.

The emulsifying polymer may be selected from the group consisting of a (hydroxypropyl)methyl cellulose, a methyl cellulose, an ethyl cellulose, an acetyl cellulose, an octenyl succinate starch, a hydroxypropyl starch, a polyvinyl pyrrolidone, a polyvinyl acetate-acrylate, a poloxamer, an albumin, a gelatin or combinations thereof.

The emulsifying polymer may be polyvinyl pyrrolidone.

The soluble polymer may be a nonionic, an anionic, a cationic, an amphoteric polymer, or a combination thereof.

The soluble polymer may be selected from the group consisting of a carboxymethyl cellulose, a carboxymethyl starch, a carboxyethyl starch, a succinyl starch, a distarch glycerol, a distarch phosphate, a hydroxypropyl distarch glycerol, a hydroxypropyl distarch phosphate, a maltodextrin, a cyclodextrin, an acacia gum, a pectin, an amylopectin, a carrageenan, a xanthan gum, a tragacanth gum, a guar gum or combination thereof.

The soluble polymer may be carboxymethyl cellulose.

The pH value may be obtained with a weak base.

The weak base may be a carbonate salt.

The carbonate salt may be selected from the group consisting of sodium carbonate (Na₂CO₃), potassium carbonate (K₂CO₃), sodium bicarbonate (NaHCO₃), and potassium and bicarbonate (KHCO₃).

The carbonate salt may be from about 1% to about 40% w/w of the delayed release dosage form or the artesunate emulsion.

The carbonate salt may be from about 5% w/w of the delayed release dosage form or the artesunate emulsion.

The delayed release dosage form may be for use for the prevention or treatment of a viral infection.

According to another embodiment, there is provided a use of artesunate or pharmaceutically acceptable salts thereof, and stereoisomers thereof for the prevention or treatment of a viral infection.

According to another embodiment, there is provided a use of artesunate or pharmaceutically acceptable salts thereof for the manufacture of a medicament for the prevention or treatment of a viral infection.

According to another embodiment, there is provided a use of a delayed release dosage form according to the present invention for the prevention or treatment of a viral infection.

According to another embodiment, there is provided a use of a delayed release dosage form according to the present invention for the manufacture of a medicament for the prevention or treatment of a viral infection.

The pharmaceutical composition for use of, the delayed release dosage form for use, or use of the present invention may further comprising the use of a second therapeutic agent.

The second therapeutic agent may be an antiviral agent, an anti-inflammatory drug, an immunomodulator, an Angiotensin Converting Enzyme (ACE) inhibitor, a chemotherapeutic agent and combinations thereof.

The antiviral agent may be Favipiravir, Abacavir, Acyclovir, Adefovir, Amantadine, Ampligen, Amprenavir, Arbidol, Atazanavir, Atripla, Balavir, Baloxavir marboxil, Biktarvy, Boceprevir, Cidofovir, Cobicistat, Combivir, Daclatasvir, Darunavir, Delavirdine, Descovy, Didanosine, Docosanol, Dolutegravir, Doravirine, Ecoliever, Edoxudine, Efavirenz, Elvitegravir, Emtricitabine, Enfuvirtide, Entecavir, Etravirine, Famciclovir, Fomivirsen, Fosamprenavir, Foscarnet, Fosfonet, Ganciclovir, Ibacitabine, Ibalizumab, Idoxuridine, Imiquimod, Imunovir, Indinavir, Inosine, an Integrase inhibitor, Interferon type I, Interferon type II, Interferon type III, Interferon, Lamivudine, Letermovir, Lopinavir, Loviride, Maraviroc, Methisazone, Moroxydine, Nelfinavir, Nevirapine, Nexavir, Nitazoxanide, Norvir, a nucleoside analogues, Oseltamivir, Peginterferon alfa-2a, Peginterferon alfa-2b, Penciclovir, Peramivir, Pleconaril, Podophyllotoxin, a protease inhibitor, Pyramidine, Raltegravir, Remdesivir, a reverse transcriptase inhibitor, Ribavirin, Rilpivirine, Rimantadine, Ritonavir, Saquinavir, Simeprevir, Sofosbuvir, Stavudine, Telaprevir, Telbivudine, Tenofovir alafenamide, Tenofovir disoproxil, Tenofovir, Tipranavir, Trifluridine, Trizivir, Tromantadine, Truvada, Umifenovir, Valaciclovir, Valganciclovir, Vicriviroc, Vidarabine, Viramidine, Zalcitabine, Zanamivir, Zidovudine, Tilorone, Mepacrine, pyronaridine and combinations thereof.

The anti-inflammatory drug may be a Non-Steroidal Anti-Inflammatory Drugs (NTHEs) comprising aspirin, acetaminophen, celecoxib, diclofenac, diflunisal, etodolac, ibuprofen, indomethacin, ketoprofen, ketorolac, nabumetone, naproxen, oxaprozin, piroxicam, salsalate, sulindac, tolmetin, indomethacin, including colchicine, and analgesic such as acetaminophene and combinations thereof.

The anti-inflammatory drug may be a corticosteroidal drug comprising Dexamethasone, Betamethasone, Prednisone, Prednisolone, Methylprednisolone, Triamcinolone, Budesonide, Flunisolide and combinations thereof.

The immunomodulator may be Anakinra, Canakinumab, Tocilizumab, Sarilumab, Baricitinib, Fedratinib, Ruxolitinib, Fingolimob, Infliximab, Adalimumab, and combinations thereof.

The ACE inhibitor may be Benazepril, Captopril, Enalapril, Fosinopril, Lisinopril, Moexipril, Perindopril, Quinapril, Ramipril, Trandolapril, and combinations thereof.

The ACE blocker may be Telmisartan, Candesartan, Irbesartan, Valsartan, Losartan, Olmesartan, Eprosartan, Azilsartan and combinations thereof.

Transmembrane Protease Serine 2 (TMPRSS2) inhibitor may be Camostat, Nafamostat, Gabexate and combination thereof.

The chemotherapeutic agent may be Daunorubicin, Mitoxantrone, Metamizole and combinations thereof.

The following terms are defined below.

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus, the phrase “consisting of” indicates that the listed elements are required or mandatory and that no other elements may be present.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “subject” is intended to mean humans and non-human mammals such as primates, cats, dogs, swine, cattle, sheep, goats, horses, rabbits, rats, mice and the like.

As used herein, the term “compound” or “compound of the present invention” is intended to mean the conjugation complex and/or the complex described herein.

As used herein, the term “pharmaceutically acceptable carrier, diluent or excipient” is intended to mean, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or encapsulating agent, such as a liposome, cyclodextrins, encapsulating polymeric delivery systems or polyethyleneglycol matrix, which is acceptable for use in the subject, preferably humans.

As used herein, the term “pharmaceutically acceptable salt” is intended to mean both acid and base addition salts.

As used herein, the term “pharmaceutically acceptable acid addition salt” is intended to mean those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

As used herein, the term “pharmaceutically acceptable base addition salt” is intended to mean those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

As used herein, the term “therapeutically effective amount” is intended to mean an amount of a compound of Formula | which, when administered to a subject is sufficient to effect treatment for a disease-state associated with insufficient apoptosis. The amount of the compound of Formula | will vary depending on the compound, the condition and its severity, and the age of the subject to be treated, but can be determined routinely by one of ordinary skill in the art having regard to his own knowledge and to this disclosure.

As used herein, the term “treating” or “treatment” is intended to mean treatment of a disease-state associated with insufficient apoptosis, as disclosed herein, in a subject, and includes: (i) preventing a disease or condition associated with insufficient apoptosis from occurring in a subject, in particular, when such mammal is predisposed to the disease or condition but has not yet been diagnosed as having it; (ii) inhibiting a disease or condition associated with insufficient apoptosis, i.e., arresting its development; or (iii) relieving a disease or condition associated with insufficient apoptosis, i.e., causing regression of the condition.

As used herein, the term “preventing disease” is intended to mean, in the case of cancer, the post-surgical, post-chemotherapy or post-radiotherapy administration of a pharmaceutical composition of the present invention to a subject, preferably a human, which was afflicted with cancer to prevent the regrowth of the cancer by killing, inhibiting the growth, or inhibiting the metastasis of any remaining cancer cells. Also included in this definition is the prevention of prosurvival conditions that lead to diseases such as asthma, MS and the like.

As used herein, the term “synergistic effect” is intended to mean that the effect achieved with the combination of the compounds of the present invention and either the chemotherapeutic agents or death receptor agonists of the invention is greater than the effect which is obtained with only one of the compounds, agents or agonists, or advantageously the effect which is obtained with the combination of the above compounds, agents or agonists is greater than the addition of the effects obtained with each of the compounds, agents or agonists used separately. Such synergy enables smaller doses to be given.

As used herein, the term “IC₅₀” is intended to mean an amount, concentration or dosage of a particular compound of the present invention that achieves a 50% inhibition of a maximal response, such as displacement of maximal fluorescent probe binding in an assay that measures such response.

As used herein, the term “EC₅₀” is intended to mean an amount, concentration or dosage of a particular compound of the present invention that achieves a 50% inhibition of cell survival.

The term “delayed release” is intended to mean that the dosage form or formulation delivers a drug (i.e. artesunate) with a delay after its administration or for a prolonged period of time, in contrast to immediate-release dosage which delivers a drug with no delay.

The compounds of the present invention, or their pharmaceutically acceptable salts may contain one or more asymmetric centers, chiral axes and chiral planes and may thus give rise to enantiomers, diastereomers, and other stereoisomeric forms and may be defined in terms of absolute stereochemistry, such as (R)—or (S)—or, as (D)- or (L)- for amino acids. The present invention is intended to include all such possible isomers, as well as, their racemic and optically pure forms. Optically active (+) and (-), (R)—and (S)—, or (D)- and (L)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively, specific enantiomers may be synthesized by asymmetric synthesis using optically active reagents, substrates, catalysts, or solvents or by converting one enantiomer to another by asymmetric transformation.

Certain compounds of the present invention may exist in Zwitterionic form and the present invention includes Zwitterionic forms of these compounds and mixtures thereof.

The compounds (in the form of the conjugation complex or the complex) of the present invention, or their salts, pharmaceutically acceptable salts or their prodrugs, may be administered in pure form or in an appropriate pharmaceutical composition, and can be carried out via any of the suitable accepted modes of Galenic pharmaceutical practice.

The pharmaceutical compositions of the present invention can be prepared by admixing a compound of the present invention with an appropriate pharmaceutically acceptable carrier, diluent or excipient, and may be formulated into preparations in solid, semi-solid, liquid form, such as tablets, capsules, powders, granules, solutions, suppositories, injections, gels, and microspheres. Typical routes of administering such pharmaceutical compositions of the present invention include oral. Pharmaceutical compositions of the present invention are formulated so as to allow the active ingredients contained therein to be bioavailable upon administration of the composition to a subject. Compositions that will be administered to a subject or patient take the form of one or more dosage units, where for example, a tablet may be a single dosage unit, and a container of a compound of the present invention in aerosol form may hold a plurality of dosage units. Actual methods of preparing such dosage forms are known, or will be apparent, to those skilled in this art; for example, see Remington’s Pharmaceutical Sciences, 18th Ed., (Mack Publishing Company, Easton, Pa., 1990). The composition to be administered will, in any event, contain a therapeutically effective amount of a compound of the present invention, or a pharmaceutically acceptable salt thereof, for treatment of a disease-state as described above.

A pharmaceutical composition of the present invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid, or an inhalable atomization or nebulization.

For oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or oil such as soybean or vegetable oil.

The pharmaceutical composition may be in the form of a liquid, e.g., an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the present invention, whether they be solutions, suspensions or other like form, may include one or more of the following adjuvants: sterile diluents such as water for injection, saline solution, preferably physiological saline, Ringer’s solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

A liquid pharmaceutical composition of the present invention used for either parenteral or oral administration should contain an amount of a compound of the present invention such that a suitable dosage will be obtained. Typically, this amount is at least 0.01% of a compound of the present invention in the composition. When intended for oral administration, this amount may be varied to be between 0.1 and about 70% of the weight of the composition. For parenteral usage, compositions and preparations according to the present invention are prepared so that a parenteral dosage unit contains between 0.01 to 1% by weight of the compound of the present invention.

The pharmaceutical composition of the present invention may include various materials, which convert the physical form of an oil liquid into solid powder form. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the present invention in solid or liquid form may include an agent that binds to the compound of the present invention and thereby assists in the delivery of the compound. Suitable agents that may act in this capacity include, but are not limited to, a monoclonal or polyclonal antibody, a protein or a liposome.

The pharmaceutical compositions of the present invention may be prepared by methodology well known in the pharmaceutical art. For example, a pharmaceutical composition intended to be administered by injection can be prepared by admixing a compound of the present invention with sterile, distilled water so as to form a solution. A surfactant may be added to facilitate the formation of a homogeneous solution or suspension. Surfactants are compounds that non-covalently interact with the compound of the present invention so as to facilitate dissolution or homogeneous suspension of the compound in the aqueous delivery system.

The compounds of the present invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose may be from about 0.1 mg to about 40 mg/kg of body weight per day or twice per day of a compound of the present invention, or a pharmaceutically acceptable salt thereof.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates the structure of a coronavirus.

FIG. 2 illustrates how IL-6/JAK/STAT and NF-κB signaling pathways targeted by viral pathogens leading a «cytokine storm» (Hypercytokinemia with increasing 11-6, IL-1β, IL-2, IL-8, IL-17, MCP-1, TNF-α, etc.).

FIG. 3 illustrates the possible mechanisms of artesunate to treat COVID-19 syndrome.

FIG. 4 illustrates the possible drugs that can be used in combination with Water-Soluble Artesunate (WSA) as therapeutic opportunities in COVID-19 patients.

FIG. 5 illustrates the FTIR analysis of WSA, artesunate, carboxymethyl cellulose and povidone.

FIG. 6A illustrates the toxicity effect of Water-Soluble Artesunate (WSA) on Vero E6 cells.

FIG. 6B illustrates the antiviral activiy of Water-Soluble Artesunate (WSA) on Vero E6 Cells.

FIG. 7 illustrates the kinetic release profile of Water-soluble artesunate (ratio 90:5:5) in simulated gastric fluid (SGF, pH 1.2) and in simulated intestinal fluid (SIF, pH 6.8).

FIG. 8 illustrates the kinetic release profile of (native) artesunate formulated with sodium carbonate in simulated gastric fluid (SGF, pH 1.2) and in simulated intestinal fluid (SIF, pH 6.8).

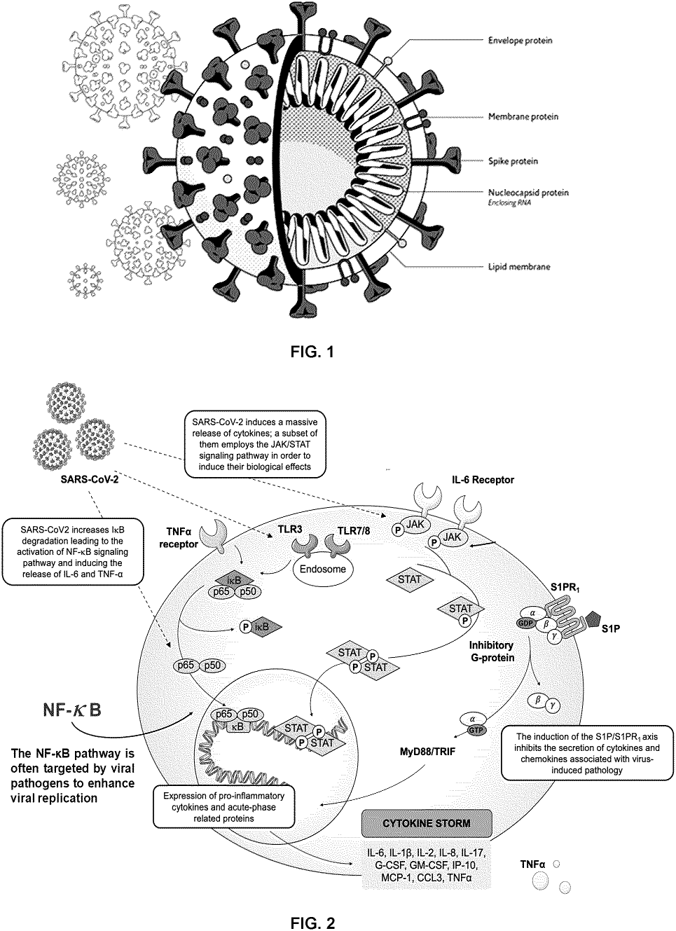

### DETAILED DESCRIPTION

### Treatment of Viral Infections Related to Coronavirus

According to an embodiment, artesunate may be used in a method of treating or preventing a viral infection in a subject in need thereof. The viral infection may be a coronavirus infection, and the coronavirus may be SARS-CoV-2.

According to an embodiment, the method of the present invention comprises administering a therapeutically effective amount of artesunate or pharmaceutically acceptable salts thereof, and stereoisomers thereof to the subject.

According to another embodiment, the method of the present invention comprises administering a therapeutically effective amount of a delayed release dosage form comprising
an artesunate or pharmaceutically acceptable salts thereof, in combination with a carbonate salt;
a therapeutically effective amount of a delayed release dosage form comprising an artesunate emulsion having a pH value of from about 7.5 to 8.0 and comprising an artesunate or pharmaceutically acceptable salts thereof, and stereoisomers thereof stabilized with an emulsifying polymer and a soluble polymer; or
a combination thereof;
to the subject.

### Artesunate Water Soluble Complex

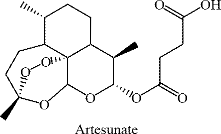

Artesunate is a hemi-succinate derivative of artemisinin. Artesunate is unstable in aqueous, acidic and basic conditions, and is sensitive to light. Also, salt forms of artesunate (such as sodium artesunate) are sticking, have low flowability, and are difficult to handle.

Commercially artesunate is available in dry powder form of artesunic acid which is poorly soluble in aqueous medium. According to the DrugBank, the water solubility of artesunic acid is about of 0.678 mg/mL (or 0.68 %, w/w). Because of this poor solubility, commercially available injection of artesunate (60 mg) requires 1 mL of sodium bicarbonate (5 % w/w) solution and dilution in 5 mL of saline (0.9 % NaCl w/w) solution immediately before use. This mode of administration is inconvenient, prone to error and could be improved. After parenteral administration, it is rapidly hydrolyzed to the active metabolite dihydroartemisinin (DHA).

For oral administration, artesunate generally remains insoluble in gastric acid and is rapidly hydrolyzed in stomach and its rate of conversion in DHA is pH dependent. Furthermore, the hydrolysis of artesunate to DHA is carried out during the stomach transit before entering the systemic circulation. Clinically, artesunate serves essentially as a prodrug for DHA. Of the current clinically used artemisinin derivatives, DHA elicits the highest neurotoxicity in cellular and animal assay. In addition, each of artesunate and DHA decomposes readily under aqueous acidic conditions to provide the inert end product 2-deoxyartemisinin. It is important to mention that only molecules with a conserved endoperoxide bridge have antimalarial activity. The other non-peroxide-containing degradation products, including 2-deoxyartemisinin, do not.

In this context, there is provided a preparation of water soluble artesunate in comparison to artesunate under salt forms and artesunate entrapped in starch glycolate known in the art (e.g. WO 2006/049391 and WO 2015/127537 respectively). This water-soluble artesunate (WSA) powder is unexpectedly stable under salt form and more soluble, not only in aqueous medium, but also in gastric acid or intestinal fluids at various temperatures. Unexpectedly, no precipitate is observed after 24 h in solution compared to commercial artesunate.

According to the present invention, water soluble artesunate may be partially converted in sodium salt form and stabilized by emulsification. This method can enhance the solubility of artesunate 100 times, and in certain case, it can reach up to 150 times. Additionally, artesunate prepared under sodium salt form and stabilized by emulsification can remain soluble in gastric acid for a long period which is suitable for delayed release formulations. Furthermore, no degradation of artesunate is detected after one-year storage under normal conditions (Temperature 22° C.±2 and Relative Humidity 40-45 %).

According to another embodiment, there is provided a delayed release dosage form comprising:

an artesunate emulsion having a pH value of from about 7.5 to 8.0 and comprising an artesunate or pharmaceutically acceptable salts thereof, and stereoisomers thereof stabilized with an emulsifying agent.

In embodiments, the weight quantity of the artesunate pharmaceutically acceptable salt in the artesunate emulsion may be from about 25% to about 90% w/w, or from about 30% to about 90% w/w, or from about 35% to about 90% w/w, or from about 40% to about 90% w/w, or from about 45% to about 90% w/w, or from about 50% to about 90% w/w, or from about 55% to about 90% w/w, or from about 60% to about 90% w/w, or from about 65% to about 90% w/w, or from about 70% to about 90% w/w, or from about 75% to about 90% w/w, or from about 80% to about 90% w/w, or from about 85% to about 90% w/w, or from about 25% to about 85% w/w, or from about 30% to about 85% w/w, or from about 35% to about 85% w/w, or from about 40% to about 85% w/w, or from about 45% to about 85% w/w, or from about 50% to about 85% w/w, or from about 55% to about 85% w/w, or from about 60% to about 85% w/w, or from about 65% to about 85% w/w, or from about 70% to about 85% w/w, or from about 75% to about 85% w/w, or from about 80% to about 85% w/w, or from about 25% to about 80% w/w, or from about 30% to about 80% w/w, or from about 35% to about 80% w/w, or from about 40% to about 80% w/w, or from about 45% to about 80% w/w, or from about 50% to about 80% w/w, or from about 55% to about 80% w/w, or from about 60% to about 80% w/w, or from about 65% to about 80% w/w, or from about 70% to about 80% w/w, or from about 75% to about 80% w/w, or from about 25% to about 75% w/w, or from about 30% to about 75% w/w, or from about 35% to about 75% w/w, or from about 40% to about 75% w/w, or from about 45% to about 75% w/w, or from about 50% to about 75% w/w, or from about 55% to about 75% w/w, or from about 60% to about 75% w/w, or from about 65% to about 75% w/w, or from about 70% to about 75% w/w, or from about 25% to about 70% w/w, or from about 30% to about 70% w/w, or from about 35% to about 70% w/w, or from about 40% to about 70% w/w, or from about 45% to about 70% w/w, or from about 50% to about 70% w/w, or from about 55% to about 70% w/w, or from about 60% to about 70% w/w, or from about 65% to about 70% w/w, or from about 25% to about 65% w/w, or from about 30% to about 65% w/w, or from about 35% to about 65% w/w, or from about 40% to about 65% w/w, or from about 45% to about 65% w/w, or from about 50% to about 65% w/w, or from about 55% to about 65% w/w, or from about 60% to about 65% w/w, or from about 25% to about 60% w/w, or from about 30% to about 60% w/w, or from about 35% to about 60% w/w, or from about 40% to about 60% w/w, or from about 45% to about 60% w/w, or from about 50% to about 60% w/w, or from about 55% to about 60% w/w, or from about 25% to about 55% w/w, or from about 30% to about 55% w/w, or from about 35% to about 55% w/w, or from about 40% to about 55% w/w, or from about 45% to about 55% w/w, or from about 50% to about 55% w/w, or from about 25% to about 50% w/w, or from about 30% to about 50% w/w, or from about 35% to about 50% w/w, or from about 40% to about 50% w/w, or from about 45% to about 50% w/w, or from about 25% to about 45% w/w, or from about 30% to about 45% w/w, or from about 35% to about 45% w/w, or from about 40% to about 45% w/w, or from about 25% to about 40% w/w, or from about 30% to about 40% w/w, or from about 35% to about 40% w/w, or from about 25% to about 35% w/w, or from about 30% to about 35% w/w, or from about 25% to about 30% w/w, or about 25% w/w, or about 26% w/w, or about 27% w/w, or about 28% w/w, or about 29% w/w, or about 30% w/w, or about 31% w/w, or about 32% w/w, or about 33% w/w, or about 34% w/w, or about 35% w/w, or about 36% w/w, or about 37% w/w, or about 38% w/w, or about 39% w/w, or about 40% w/w, or about 41% w/w, or about 42% w/w, or about 43% w/w, or about 44% w/w, or about 45% w/w, or about 46% w/w, or about 47% w/w, or about 48% w/w, or about 49% w/w, or about 50% w/w, or about 51% w/w, or about 52% w/w, or about 53% w/w, or about 54% w/w, or about 55% w/w, or about 56% w/w, or about 57% w/w, or about 58% w/w, or about 59% w/w, or about 60% w/w, or about 61% w/w, or about 62% w/w, or about 63% w/w, or about 64% w/w, or about 65% w/w, or about 66% w/w, or about 67% w/w, or about 68% w/w, or about 69% w/w, or about 70% w/w, or about 71% w/w, or about 72% w/w, or about 73% w/w, or about 74% w/w, or about 75% w/w, or about 76% w/w, or about 77% w/w, or about 78% w/w, or about 79% w/w, or about 80% w/w, or about 81% w/w, or about 82% w/w, or about 83% w/w, or about 84% w/w, or about 85% w/w, or about 86% w/w, or about 87% w/w, or about 88% w/w, or about 89% w/w, or about 90% w/w of the total weight of the emulsion.

In embodiments, the weight quantity of the emulsifying polymer and/or the soluble polymer in the artesunate emulsion may be from about 1% to about 55% w/w, or about 2% to about 55% w/w, or about 3% to about 55% w/w, or about 4% to about 55% w/w, or about 5% to about 55% w/w, or about 6% to about 55% w/w, or about 7% to about 55% w/w, or about 8% to about 55% w/w, or about 9% to about 55% w/w, or about 10% to about 55% w/w, or from about 15% to about 55% w/w, or from about 20% to about 55% w/w, or from about 25% to about 55% w/w, or from about 30% to about 55% w/w, or from about 35% to about 55% w/w, or from about 40% to about 55% w/w, or from about 45% to about 55% w/w, or from about 1% to about 50% w/w, or about 2% to about 50% w/w, or about 3% to about 50% w/w, or about 4% to about 50% w/w, or about 5% to about 50% w/w, or about 6% to about 50% w/w, or about 7% to about 50% w/w, or about 8% to about 50% w/w, or about 9% to about 50% w/w, or about 10% to about 50% w/w, or from about 15% to about 50% w/w, or from about 20% to about 50% w/w, or from about 25% to about 50% w/w, or from about 30% to about 50% w/w, or from about 35% to about 50% w/w, or from about 40% to about 50% w/w, or from about 45% to about 50% w/w, or from about 1% to about 45% w/w, or about 2% to about 45% w/w, or about 3% to about 45% w/w, or about 4% to about 45% w/w, or about 5% to about 45% w/w, or about 6% to about 45% w/w, or about 7% to about 45% w/w, or about 8% to about 45% w/w, or about 9% to about 45% w/w, or about 10% to about 45% w/w, or from about 15% to about 45% w/w, or from about 20% to about 45% w/w, or from about 20% to about 45% w/w, or from about 25% to about 45% w/w, or from about 30% to about 45% w/w, or from about 35% to about 45% w/w, or from about 40% to about 45% w/w, or from about 1% to about 40% w/w, or about 2% to about 40% w/w, or about 3% to about 40% w/w, or about 4% to about 40% w/w, or about 5% to about 40% w/w, or about 6% to about 40% w/w, or about 7% to about 40% w/w, or about 8% to about 40% w/w, or about 9% to about 40% w/w, or about 10% to about 40% w/w, or from about 15% to about 40% w/w, or from about 20% to about 40% w/w, or from about 25% to about 40% w/w, or from about 30% to about 40% w/w, or from about 35% to about 40% w/w, or from about 1% to about 35% w/w, or about 2% to about 35% w/w, or about 3% to about 35% w/w, or about 4% to about 35% w/w, or about 5% to about 35% w/w, or about 6% to about 35% w/w, or about 7% to about 35% w/w, or about 8% to about 35% w/w, or about 9% to about 35% w/w, or about 10% to about 35% w/w, or from about 15% to about 35% w/w, or from about 20% to about 35% w/w, or from about 25% to about 35% w/w, or from about 30% to about 35% w/w, or from about 1% to about 30% w/w, or about 2% to about 30% w/w, or about 3% to about 30% w/w, or about 4% to about 30% w/w, or about 5% to about 30% w/w, or about 6% to about 30% w/w, or about 7% to about 30% w/w, or about 8% to about 30% w/w, or about 9% to about 30% w/w, or about 10% to about 30% w/w, or from about 15% to about 30% w/w, or from about 20% to about 30% w/w, or from about 25% to about 30% w/w, or from about 1% to about 25% w/w, or about 2% to about 25% w/w, or about 3% to about 25% w/w, or about 4% to about 25% w/w, or about 5% to about 25% w/w, or about 6% to about 25% w/w, or about 7% to about 25% w/w, or about 8% to about 25% w/w, or about 9% to about 25% w/w, or about 10% to about 25% w/w, or from about 15% to about 25% w/w, or from about 20% to about 25% w/w, or from about 1% to about 20% w/w, or about 2% to about 20% w/w, or about 3% to about 20% w/w, or about 4% to about 20% w/w, or about 5% to about 20% w/w, or about 6% to about 20% w/w, or about 7% to about 20% w/w, or about 8% to about 20% w/w, or about 9% to about 20% w/w, or about 10% to about 20% w/w, or from about 15% to about 20% w/w, or from about 1% to about 15% w/w, or about 2% to about 15% w/w, or about 3% to about 15% w/w, or about 4% to about 15% w/w, or about 5% to about 15% w/w, or about 6% to about 15% w/w, or about 7% to about 15% w/w, or about 8% to about 15% w/w, or about 9% to about 15% w/w, or about 10% to about 15% w/w, or from about 1% to about 10% w/w, or about 2% to about 10% w/w, or about 3% to about 10% w/w, or about 4% to about 10% w/w, or about 5% to about 10% w/w, or about 6% to about 10% w/w, or about 7% to about 10% w/w, or about 8% to about 10% w/w, or about 9% to about 10% w/w, or from about 1% to about 2% w/w, or about 1% to about 5% w/w, or from about 1% w/w, or from about 2% w/w, or from about 3% w/w, or from about 4% w/w, or from about 5% w/w, or from about 6% w/w, or from about 7% w/w, or from about 8% w/w, or from about 9% w/w, or from about 10% w/w, or from about 11% w/w, or from about 12% w/w, or from about 13% w/w, or from about 14% w/w, or from about 15% w/w, or from about 16% w/w, or from about 17% w/w, or from about 18% w/w, or from about 19% w/w, or from about 20% w/w, or from about 21% w/w, or from about 22% w/w, or from about 23% w/w, or from about 24% w/w, or from about 25% w/w, or from about 26% w/w, or from about 27% w/w, or from about 28% w/w, or from about 29% w/w, or from about 30% w/w, or from about 31% w/w, or from about 32% w/w, or from about 33% w/w, or from about 34% w/w, or from about 35% w/w, or from about 36% w/w, or from about 37% w/w, or from about 38% w/w, or from about 39% w/w, or from about 40% w/w, or from about 41% w/w, or from about 42% w/w, or from about 43% w/w, or from about 44% w/w, or from about 45% w/w, or from about 46% w/w, or from about 47% w/w, or from about 48% w/w, or from about 49% w/w, or from about 50% w/w, or from about 51% w/w, or from about 52% w/w, or from about 53% w/w, or from about 54% w/w, or from about 55% w/w, of each of the emulsifying polymer and/or the soluble polymer.

In embodiments, preferred weight ratios of the artesunate pharmaceutically acceptable salt, the emulsifying polymer and the soluble polymer in the artesunate emulsion may be from about (artesunate : emulsifying polymer : soluble polymer) 25:37.5:37.5; 80:10:10, 90:8:2 and 90:5:5 w/w.

### Soluble Polymers

The soluble polymers may be an anionic, a nonionic, a cationic, an amphoteric soluble polymer, or a combination thereof. The soluble polymer may be selected from the group consisting of carboxymethyl cellulose (CMC), carboxymethyl starch (CMS), carboxyethyl starch (CES), succinyl starch (SS), distarch glycerol (DG), distarch phosphate (DP), hydroxypropyl distarch glycerol (HPDG), hydroxypropyl distarch phosphate (HPDP), maltodextrin, cyclodextrin, acacia gum, pectin, amylopectin, carrageenan, xanthan gum, tragacanth gum, guar gum or combination thereof.

### Emulsifying Polymers

The emulsifying polymer may be an anionic, a nonionic, a cationic, an amphoteric emulsifying polymer, or a combination thereof. The emulsifying polymer may be selected from the group consisting of (hydroxypropyl)methyl cellulose (HPMC), methyl cellulose (MC), ethyl cellulose (EC), acetyl cellulose (AcC), octenyl succinate starch, hydroxypropyl starch, polyvinyl pyrrolidone, polyvinyl acetate-acrylate, poloxamer, albumin, gelatin or combination thereof.

According to another embodiment, the pH value may be obtained with a weak base. For example, the weak base may be a carbonate salt.

In the present invention, the carbonate salts may be salts such as sodium carbonate (Na₂CO₃), potassium carbonate (K₂CO₃), sodium bicarbonate (NaHCO₃), and potassium bicarbonate (KHCO₃), magnesium carbonate (MgCO₃), and calcium carbonate (CaCO₃) and combinations thereof. They may be used with an artesunate or pharmaceutically acceptable salts thereof, and/or in combination with an artesunate or pharmaceutically acceptable salts thereof and the emulsifying polymer and the soluble polymer. In embodiments, the carbonate salt may be from about 1% to about 40%, or from about 2% to about 40%, or from about 3% to about 40%, or from about 4% to about 40%, or from about 5% to about 40%, or from about 10% to about 40%, or from about 15% to about 40%, or from about 20% to about 40%, or from about 25% to about 40%, or from about 30% to about 40%, or from about 35% to about 40%, or from about 1% to about 35%, or from about 2% to about 35%, or from about 3% to about 35%, or from about 4% to about 35%, or from about 5% to about 35%, or from about 10% to about 35%, or from about 15% to about 35%, or from about 20% to about 35%, or from about 25% to about 35%, or from about 30% to about 35%, or from about 1% to about 30%, or from about 2% to about 30%, or from about 3% to about 30%, or from about 4% to about 30%, or from about 5% to about 30%, or from about 10% to about 30%, or from about 15% to about 30%, or from about 20% to about 30%, or from about 25% to about 30%, or from about 1% to about 25%, or from about 2% to about 25%, or from about 3% to about 25%, or from about 4% to about 25%, or from about 5% to about 25%, or from about 10% to about 25%, or from about 15% to about 25%, or from about 20% to about 25%, or from about 1% to about 20%, or from about 2% to about 20%, or from about 3% to about 20%, or from about 4% to about 20%, or from about 5% to about 20%, or from about 10% to about 20%, or from about 15% to about 20%, or from about 1% to about 15%, or from about 2% to about 15%, or from about 3% to about 15%, or from about 4% to about 15%, or from about 5% to about 15%, or from about 10% to about 15%, or from about 1% to about 10%, or from about 2% to about 10%, or from about 3% to about 10%, or from about 4% to about 10%, or from about 5% to about 10%, or from about 1% to about 5%, or from about 2% to about 5%, or from about 3% to about 5%, or from about 4% to about 5%, or from about 1% to about 4%, or from about 2% to about 4%, or from about 3% to about 4%, or from about 1% to about 3%, or from about 2% to about 3%, or from about 1% to about 2%, or about 1%, or about 2%, or about 3%, or about 4%, or about 5%, or about 6%, or about 7%, or about 8%, or about 9%, or about 10%, or about 11%, or about 12%, or about 13%, or about 14%, or about 15%, or about 16%, or about 17%, or about 18%, or about 19%, or about 20%, or about 21%, or about 22%, or about 23%, or about 24%, or about 25%, or about 26%, or about 27%, or about 28%, or about 29%, or about 30%, or about 31%, or about 32%, or about 33%, or about 34%, or about 35%, or about 36%, or about 37%, or about 38%, or about 39%, or about 40% w/w of the delayed release dosage form

### Use of the Composition of the Present Invention

According to an embodiment, artesunate formulated as pharmaceutical compositions, and in particular the water soluble artesunate of the present invention may be used for the treatment of inflammation, particularly inflammation related to coronavirus infection and most particularly the respiratory syndrome associated with coronavirus, such as COVID-19.

According to an embodiment, there is provided a method for prevention or treatment of viral infection, particularly coronavirus infection such as the SARS-CoV-2 virus causing COVID-19, comprising administering to a subject in need thereof a therapeutically effective amount of artesunate or a pharmaceutically acceptable salt thereof.

Also, according to another embodiment, there is provided the use of artesunate or a pharmaceutically acceptable salt thereof for the prevention or treatment of a viral infection, particularly coronavirus infection such as the SARS-CoV-2 virus causing COVID-19 in a subject in need thereof.

Also, according to another embodiment, there is provided the use of artesunate or a pharmaceutically acceptable salt thereof, for the preparation of a medicament for the prevention or treatment of viral infection, particularly coronavirus infection such as the SARS-CoV-2 virus causing COVID-19.

Also, according to another embodiment, there is provided a pharmaceutical composition comprising artesunate or a pharmaceutically acceptable salt thereof for use for the prevention or treatment of viral infection, particularly coronavirus infection such as the SARS-CoV-2 virus causing COVID-19.

According to an embodiment, there is provided a method for prevention or treatment of viral infection, particularly coronavirus infection such as the SARS-CoV-2 virus causing COVID-19, comprising administering to a subject in need thereof a therapeutically effective amount of the delayed release dosage form of the present invention.

Also, according to another embodiment, there is provided the use of a delayed release dosage form of the present invention for the prevention or treatment of a viral infection, particularly coronavirus infection such as the SARS-CoV-2 virus causing COVID-19 in a subject in need thereof.

Also, according to another embodiment, there is provided the use of a delayed release dosage form of the present invention for the preparation of a medicament for the prevention or treatment of viral infection, particularly coronavirus infection such as the SARS-CoV-2 virus causing COVID-19.

Also, according to another embodiment, there is provided a delayed release dosage form of the present invention for use for the prevention or treatment of viral infection, particularly coronavirus infection such as the SARS-CoV-2 virus causing COVID-19.

### Coronavirus, Covid-19 and Immune Response

### IL-6/JAK/STAT Signaling Pathway

The JAK/STAT signaling is a key pathway transducing extracellular signals transmitted by many cytokines, lymphokines and growth factors. Particularly, a subset of cytokines employs the JAK/STAT signaling pathway to induce their biological effects. Notably, one of the major activators of JAK/STAT signaling is the cytokine IL-6, which has been reported to be dramatically increased in COVID-19 patients, with a strong implication in acute inflammation and cytokine storm. Accordingly, the synthesis and secretion of IL-6 has been demonstrated to be induced by angiotensin II, which is locally produced by the inflamed vessels in a JAK/STAT-dependent manner. In particular, angiotensin II binding to Angiotensin II receptor type 1 (AT1 receptor) has been found to activate JAK/STAT pathway and to promote the downstream production of IL-6. Increased angiotensin II enhances IL-6 production in AT1/JAK/STAT-dependent manner, thus establishing a positive inflammatory feedback loop. Interestingly, the spike protein of SARS-CoV has been demonstrated to downregulate ACE2 expression, thus resulting in over-production of angiotensin II by the related enzyme ACE. In a similar way, it could be hypothesized that SARS-CoV-2 may downregulate ACE2 receptors, thus leading to an over-production of angiotensin II, in turn enhancing IL-6 production in AT1/JAK/STAT and ultimately driving to vascular inflammation and lung injury, clinical signatures of COVID-19. Moreover, the angiotensin II/AT1 receptor axis has been reported to also activate both NF-κB.

### NF-κB Signaling Pathway

The NF-κB signaling pathway is often targeted by viral pathogens to enhance viral replication, host cell survival and host immune evasion. Viruses may activate or suppress NF-κB.

Since SARS-CoV-2 (causing COVID-19) belongs to the same family of corona viruses and it shares many similarities. SARS-CoV-2 activates NF-κB pathway, like MERS and SARS-CoV before it. SARS-CoV viruses appear to promote inflammatory mediators in vitro and in vivo through actions on NF-κB. Levels of NF-κB are higher in lungs of recombinant SARS (rSARS)-infected mice. Inhibitors of NF-κB reduced rSARS-CoV-induced inflammation. NF-κB is specifically induced by SARS-CoV S protein to produce inflammatory mediators that are associated with ARDS in SARS in vitro.

The main management of COVID-19 is the respiratory failure from acute respiratory distress syndrome which causes of the mortality. Additionally, hemophagocytic lymphohistiocytosis is also manifested by a hyperinflammatory syndrome characterized by a fulminant and fatal hypercytokinemia with multiorgan failure. This severe hyperinflammation is generally caused by uncontrolled proliferation of activated lymphocytes and macrophages that secrete high amounts of inflammatory cytokines, hence the appellation «cytokine storm syndrome». The hypercytokinemia associated with COVID-19 disease severity is characterized by increased interleukin (IL) including IL-2, IL-7, granulocyte-colony stimulating factor (G-CSF), interferon-γ inducible protein, monocyte chemoattractant protein, macrophage inflammatory protein 1-α, and tumor necrosis factor-α (TNF-α).

A controlled trial consisting of blocking IL-1 in sepsis and IL-6 receptor (licensed for cytokine release syndrome) showed significant survival benefit in patients with hyperinflammation, without increased adverse events. It is important to mention that Janus kinase (JAK) inhibition could also affect both inflammation and cellular viral entry in COVID-19. Therapeutic options are of interest to prevent the cytokine storm (hypercytokinemia) include selective cytokine blockade and JAK inhibition.

### Management of Coronavirus (COVID-19) Infection With Artesunate

Artesunate is a semisynthetic derivative of artemisinin that is isolated from plant Artemisa annua, sweet wormwood. This herb employed in Chinese traditional medicine used against malaria due to Plasmodium falciparum. Artemisinin is a sesquiterpene lactone containing an unusual peroxide bridge which is responsible for the drug’s mechanism of action. Because the physical properties of artemisinin itself such as poor solubility, low bioavailability limited its effectiveness, semisynthetic derivatives of artemisinin have been developed. It is important to mention artesunate, arteether and artemether. These derivatives are mostly applied through the digestive tract, either by oral or rectal administration. However, artesunate is the only compound available for intravenously, oral administration including sublingual or buccal dosage form, etc.

### Safety Profile of Artesunate

As mentioned previously, artesunate is used in monotherapy to treat malaria. Due to appearance of resistance forms, artesunate is preferably combined with another antimalarial drug such as artesunate and amodiaquine (Amino-4-quinoline) Winthrop developed by Sanofi-Aventis.

To address the immediate need to reduce the rising mortality caused by COVID-19, it is preferable to use existing approved drugs with proven safety profiles. Artesunate is fitting well for the present case because it is safe, low cost drug which has been used by thousands of patients with malaria without important adverse effects. Additionally, Artesunate is recommended by the World Health Organization (WHO) as the treatment of choice for severe malaria. Although the mechanism behind the antimalarial activity of artesunate is not entirely clear, it is thought that the active metabolite, Dihydroartemisinin (DHA) is responsible for the death of the parasite.

### Activity of Artesunate Against COVID-19

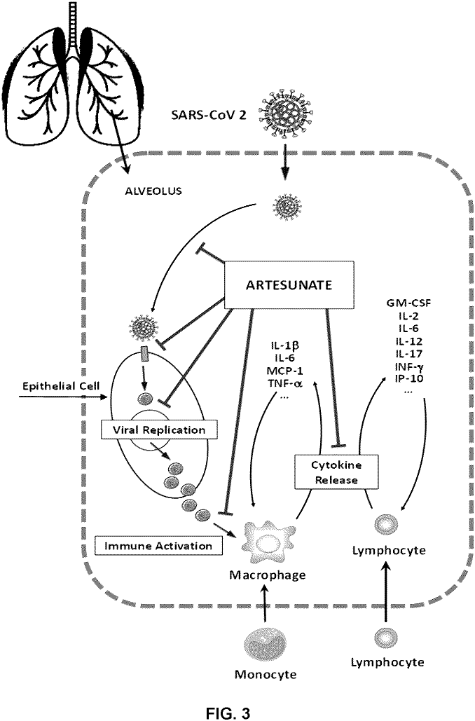

Artesunate possesses anti-viral activity due to its endoperoxide bridge. In addition, it also possesses anti-inflammatory properties due to its blockade of the production of interleukins (IL)-1β, IL-6 and IL-8 in tumor necrosis factor-α (TNF-α) pathway via the regulation of NF-κB and phosphoinositide 3 kinase (Pl3K) pathways. (See FIG. 3).

It is of interest to mention that artesunate can regulate the expression of pro-inflammatory cytokines, nuclear factor-kappa B (NF-κB), matrix metalloproteinases (MMPs), vascular endothelial growth factor (VEGF), promote cell cycle arrest and drive reactive oxygen species (ROS) production. Artesunate also possesses potent and broad antibacterial, anticancer and particularly antiviral properties. It is worthy to mention that Artesunate may also exert powerful anti-inflammatory effects in experimental asthma, pancreatitis, arthritis, and sepsis.

With regard of antiviral properties, artesunate includes the inhibition of several viruses, such as human cytomegalovirus and other members of the Herpesviridae family (e.g., herpes simplex virus type 1 and Epstein-Barr virus), hepatitis B virus, hepatitis C virus, and bovine viral diarrhea virus. Artesunate could inhibited central regulatory processes of virus-infected cells (such as activation pathways dependent on NF-κB), thus interfering with critical host-cell-type and metabolism requirements for virus replication. It is also shown that Hemin, an iron donor, in combination with artesunate inhibits Hepatitis C virus replication by inhibiting the viral polymerase.

Artesunate can be considered a powerful anti-inflammatory. It has been suggested that this anti-inflammatory effect is due to the inhibition of the expression and the release of pro-inflammatory cytokines in the lung tissues. This property is suggesting that the use artesunate for treatment of coronavirus pneumonia syndrome may be efficient. The anti-inflammatory activity may be due to the inhibition of the proinflammatory cytokine production via the blockade of NF-_{K}B and PI3 kinase/Akt signal pathway. Artesunate may inhibit Akt (protein kinase B) activation and consequently prevent IL-1, IL-6 and IL-8 production. Artesunate could also inhibit the Janus Kinase/Signal Transducer and Transcription Activator (JAK/STAT) signaling pathway, thus resulting in inhibition of the pro-inflammatory cytokines, TNF-α, IL-6, IL-8 and monocyte chemoattractant protein (MCP-) 1.

Without wishing to be bound by theory, it is believed that the use of artesunate to treat the inflammation is an advantageous option, particularly to provide relief in the case of coronavirus pneumonia syndrome. This anti-inflammatory activity could be exerted via various pathways. One mechanism may be the inhibition of the JAK/STAT signaling pathway to prevent the production of the pro-inflammatory cytokines such as TNF-α, IL-6, IL-8 and MCP-1 (FIG. 3). Other anti-inflammatory mechanisms are also demonstrated. Artesunate can inhibit inflammation by reducing prostaglandin E2 production (involved in inflammation) and by inhibiting a range of proinflammatory molecules, including interleukin-1 receptor-associated kinase 1 (IRAK1), Forkhead Box O4 (FOXO4), glycogen synthase kinase-3b (GSK-3b), and the transcription NF-_{K}B, for example.

In addition, clinical evidence suggests that the intestine may present another target organ for coronavirus. As highlighted by several studies, Angiotensin Converting Enzyme-2 (ACE-2) is an entry receptor for SARS-CoV-2. ACE-2 is highly expressed on differentiated enterocytes. In human small intestinal organoids, enterocytes were readily infected by SARS-CoV and SARS-CoV-2. Hence, intestinal epithelium supports SARS-CoV-2 replication. Artesunate possesses an endoperoxide molecule able to generate reactive oxygen species (ROS). Artesunate treatment for 24h causes a significant increase in the levels of ROS in a dose-dependent manner in cell lines. For this reason, artesunate can generate ROS and inactivate coronavirus by damage directly to the vital cellular structure of coronavirus such as its genome (RNA), its nucleocapsid protein and membrane or envelope protein, for example.

Therefore, artesunate as treatment for COVID-19 would exert its action against coronavirus in different ways: inhibition of JAK/STAT signaling pathway, which can affect both inflammation and cellular entry in Covid-19; suppression of expression of pro-inflammatory cytokines TNF-α, IL-6, IL-8 and MCP-1; blockade of the PI3K/Akt signaling pathway; regulation of NF-_{K}B signaling pathway; and by generating ROS which attack on the viral structure.

### Combination Therapy

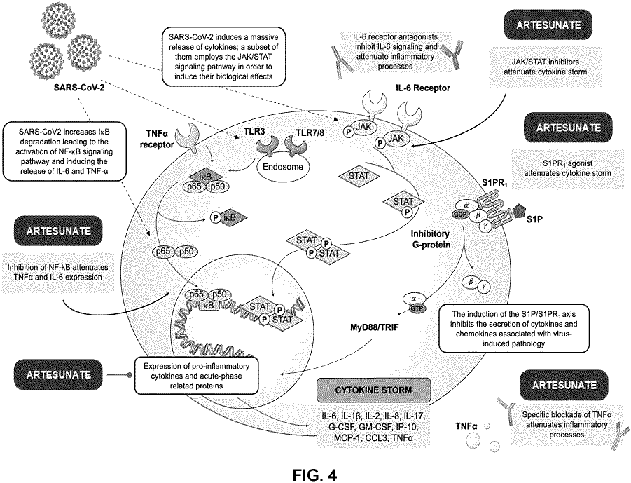

Furthermore, it is of interest to use a combination of therapy along with artesunate treatment. Combination of drugs with the Artesunate may offer complete and rapid relieve acute respiratory distress syndrome in Covid-19 patients and thus increase the chance of survival for Covid-19 patients. In fact, now referring to FIG. 4, artesunate may be combined for therapy to provide numerous advantages: reach different targets and relieve symptoms caused by coronavirus rapidly; improve the effectiveness of treatment, for example, artesunate can be combined with selective cytokine inhibitors such as Anakinra™ (to block the inflammatory protein IL-1) and/or Tocilizumab (to block IL-6), thus increasing the beneficial effects.

To increase the effectiveness and reduce the adverse effects, the water soluble artesunate could be combined with different drugs. Repurposing different FDA-approved drugs could be a fast way and a better option to get treatment to patients promptly.

Drugs may include antiviral agents such as Favipiravir, Abacavir (Use for HIV), Acyclovir (Use for herpes e.g. Chicken pox), Adefovir (Use for chronic Hepatitis B), Amantadine (Use for influenza), Ampligen, Amprenavir (Use for inhibition of HIV), Arbidol, Atazanavir, Atripla (fixed dose drug), Balavir, Baloxavir marboxil, Biktarvy, Boceprevir, Cidofovir, Cobicistat, Combivir, Daclatasvir, Darunavir, Delavirdine, Descovy, Didanosine, Docosanol, Dolutegravir, Doravirine, Ecoliever, Edoxudine, Efavirenz, Elvitegravir, Emtricitabine, Enfuvirtide, Entecavir, Etravirine, Famciclovir, Fomivirsen, Fosamprenavir, Foscarnet, Fosfonet, Ganciclovir, lbacitabine, lbalizumab, Idoxuridine, Imiquimod, Imunovir, Indinavir, Inosine, an Integrase inhibitor, Interferon type I, Interferon type II, Interferon type III, Interferon, Lamivudine, Letermovir, Lopinavir, Loviride, Maraviroc, Methisazone, Moroxydine, Nelfinavir, Nevirapine, Nexavir, Nitazoxanide, Norvir, a nucleoside analogues, Oseltamivir, Peginterferon alfa-2a, Peginterferon alfa-2b, Penciclovir, Peramivir, Pleconaril, Podophyllotoxin, a protease inhibitor, Pyramidine, Raltegravir, Remdesivir, a reverse transcriptase inhibitor, Ribavirin, Rilpivirine, Rimantadine, Ritonavir, Saquinavir, Simeprevir, Sofosbuvir, Stavudine, Telaprevir, Telbivudine, Tenofovir alafenamide, Tenofovir disoproxil, Tenofovir, Tipranavir, Trifluridine, Trizivir, Tromantadine, Truvada, Umifenovir, Valaciclovir, Valganciclovir, Vicriviroc, Vidarabine, Viramidine, Zalcitabine, Zanamivir, Zidovudine Tilorone, Mepacrine, pyronaridine and combinations thereof.

The anti-inflammatory drug may be Non-Steroidal Anti-Inflammatory Drugs (NSAID) such as aspirin, celecoxib, diclofenac, diflunisal, etodolac, ibuprofen, indomethacin, ketoprofen, ketorolac, nabumetone, naproxen, oxaprozin, piroxicam, salsalate, sulindac, tolmetin, indomethacin, including colchicine, and analgesic such as acetaminophen and combinations thereof.

The anti-inflammatory drug may be Steroid (corticosteroid) such as Dexamethasone, Betamethasone, Prednisone, Prednisolone, Methylprednisolone, Triamcinolone, Budesonide, Flunisolide and combinations thereof.

Drugs may also include immunomodulator (cytokine inhibitors) such as Anakinra, Canakinumab, Tocilizumab, Sarilumab, Baricitinib, Fedratinib, Ruxolitinib, Fingolimob, Infliximab, and Adalimumab.

Drugs may also include Angiotensin Converting Enzyme (ACE) inhibitor such as Benazepril, Captopril, Enalapril, Fosinopril, Lisinopril, Moexipril, Perindopril, Quinapril, Ramipril, and Trandolapril.

The ACE blocker may be Telmisartan, Candesartan, Irbesartan, Valsartan, Losartan, Olmesartan, Eprosartan, Azilsartan and combinations thereof.

Transmembrane Protease Serine 2 (TMPRSS2) inhibitor may be Camostat, Nafamostat, Gabexate and combination thereof.

Drugs may also include chemotherapeutic agents such as Daunorubicin, Mitoxantrone, Metamizole.

Recently, the role of vitamin D (Cholecalciferol) in regulating the immune system is supported by multiple studies. Therefore, according to an embodiment, the drug may also include vitamin D, which is believed to be able to suppress cytokine production by simultaneously boosting the innate immune system and avoiding the overactivation of the adaptive immune system to immediately respond to viral load. For this reason, Vitamin D can also be administered in combination with Artesunate.

### Posology and Method of Administration

Since WSA could be administrated intravenously (IV) or orally, there are two dosage forms are prepared for such purposes, mainly: powder dosage form in vial 60 mg for IV administration and, monolithic tablet dosage form 200 mg for oral administration.

### For IV Administration

The maximal daily dose is 120 mg b.i.d. (60 mg x 2 vials) for 7-14 days. For IV administration, inject 5 mL of saline (0.9 % NaCl) solution into vial to dissolve WSA until obtaining a clear solution before injection.

### For Oral Administration

The daily dose recommended for treatment is 100-200 mg b.i.d. (50-100 mg x2 tablets) or 150-300 t.i.d. (x 3 tablets), but not to exceed 400 mg/day. In combination therapy, WSA can be administered separately before or after, or at the same time with other drugs.

The composition of the present invention may be useful in the prevention and/or treatment of viral infection, particularly coronavirus infection such as the SARS-CoV-2 virus causing COVID-19.

### Process for Preparation of Water Soluble Artesunate

According to another embodiment, there is disclosed a process for the preparation of a delayed release formulation comprising an artesunate emulsion comprising the steps of:
a) introducing an artesunate or pharmaceutically acceptable salts thereof, or stereoisomers thereof in a buffered solution comprising an emulsifying polymers and soluble polymers, to obtain an emulsified artesunate solution;
b) adjusting pH value of the emulsified artesunate solution to a pH value of about 7.5 to about 8.0, to obtain an adjusted emulsified artesunate solution; and
c) drying the adjusted emulsified artesunate solution, to obtain a dry powder of artesunate.

The pH value of the dry powder of emulsified artesunate may be from about 7.5 to about 8.0, or about 7.6 to about 8.0, or about 7.7 to about 8.0, or about 7.8 to about 8.0, or about 7.9 to about 8.0, 7.5 to about 7.9, or from about 7.6 to about 7.9, or from about 7.7 to about 7.9, or from about 7.8 to about 7.9, or from about 7.5 to about 7.8, or from about 7.6 to about 7.8, or from about 7.7 to about 7.8, or from about 7.5 to about 7.7, or from about 7.6 to about 7.7, or from about 7.5 to about 7.6, or about 7.5, 7.55, 7.6, 7.65, 7.7, 7.75, 7.8, 7.85, 7.9, or 8.0.

The weight ratio of the artesunate pharmaceutically acceptable salt and the emulsifying agent is from about 9:1 to about 1:1 (see also the enumeration above). Preferably, the ratio is 3:2.

### Soluble Polymers

The soluble polymers may be an anionic, a nonionic, a cationic, an amphoteric soluble polymer, or a combination thereof. The soluble polymer may be selected from the group consisting of carboxymethyl cellulose (CMC), carboxymethyl starch (CMS), carboxyethyl starch (CES), succinyl starch (SS), distarch glycerol (DG), distarch phosphate (DP), hydroxypropyl distarch glycerol (HPDG), hydroxypropyl distarch phosphate (HPDP), maltodextrin, cyclodextrin, acacia gum, pectin, amylopectin, carrageenan, xanthan gum, tragacanth gum, guar gum or combination thereof.

### Emulsifying Polymers

The emulsifying polymer may be an anionic, a nonionic, a cationic, an amphoteric emulsifying polymer, or a combination thereof. The emulsifying polymer may be selected from the group consisting of (hydroxypropyl)methyl cellulose (HPMC), methyl cellulose (MC), ethyl cellulose (EC), acetyl cellulose (AcC), octenyl succinate starch, hydroxypropyl starch, polyvinyl pyrrolidone, polyvinyl acetate-acrylate, poloxamer, albumin, gelatin or combination thereof.

According to another embodiment, the pH value may be obtained with a weak base. For example, the weak base may be a carbonate salt. In the present invention, the carbonate salts may be salts such as sodium carbonate (Na₂CO₃), potassium carbonate (K₂CO₃), sodium bicarbonate (NaHCO₃), and potassium bicarbonate (KHCO₃), magnesium carbonate (MgCO₃), and calcium carbonate (CaCO₃) and combinations thereof.

The drying may be by spray drying.

The present invention will be more readily understood by referring to the following examples which are given to illustrate the invention rather than to limit its scope.

### Example 1

### Preparation of Artesunate

### Preparation of Artesunate Under Sodium Carbonate Salt Form

An amount of 18 g of artesunate is dispersed in 54 mL of water and 33 mL of aqueous sodium bicarbonate solution (4.16 %) is then added. The solution is stirred mildly for 20 minutes at 25° C., filtered and then freeze-dried to provide a white crystalline powder.

### Immobilisation of Artesunate in Starch Glycolate by Entrapment

An amount of 10 g of water soluble artesunate is dispersed in 50 mL of ethanol and homogenized until a clear solution is obtained. The solution is sprayed directly on the surface of the starch glycolate powder (40 g) using an atomizer, similarly to a fluid bed granulation process.

### Preparation of Water-Soluble Artesunate Solid Powder

Different weight ratio of artesunate and emulsifying agent can be prepared, for example from 9:1 to 1:1. Preferably, the artesunate / emulsifying polymer / soluble polymer weight ratio is about 90:5:5. To prepare this formulation of water-soluble artesunate solid powder forms in the present invention, the following steps may be performed.

### Materials

Artesunate was purchased from HUVEPHARMA Italia S.R.L (Garessio, CN, Italy);
Carboxymethyl cellulose (low viscosity);
Kollidon® 12 PF (Povidone or polyvinyl pyrrolidone) was from BASF (67056, Ludwigshafen, Germany);
Sodium Carbonate (anhydrous, under powder form) was from SPECTRUM Chemical MFG Corp (755 Jersey Avenue, New Brunswick, NJ, USA);
Sterile distilled water.

### Preparation of Water-Soluble Artesunate Spray-Drying Solution

Disperse an amount of 2.5 g of sodium carboxymethyl cellulose in 300 mL purified water under mild stirring at room temperature until obtaining a clear solution;
Add 2.5 g of Kollidon® 12PF under similar conditions into the solution;
Adjust the pH of solution at 7.9±0.1 with sodium carbonate;
Introduce slowly 10.57 g of sodium carbonate, always under mild stirring;
Dissolve 45 g of Artesunic acid (commercial Artesunate) in the solution and homogenize until the solution is clear;
Adjust the pH of solution at 7.9±0.1;
Complete the solution with purified water at 375 mL.

An Artesunate:Carboxymethylcellulose:Kollidon weight ratio represents approximately 90:5:5.

Unexpectedly, the pH of the solution is very important. The acceptable pH values should be between 7.5 - 8.0, and preferably 7.6-7.8. Artesunate powder prepared at pH higher than 8.0 is sticking and has poor flowability, particularly when the drying is performed in a spray-dryer. In this case, it may be difficult to fill completely the artesunate powder quantity in the die to obtain tablets by compaction. The pH value of 7.4 or lower provide artesunate powders presenting good physical properties, but poorly soluble.

Weak bases are preferable to increase the pH of the artesunate solution to avoid the degradation of artesunate, because Artesunic acid is very soluble in alkaline solutions, but hydrolyses rapidly to DHA. Generally, carbonate salts such as sodium (or potassium) carbonate (Na₂CO₃) or sodium (or potassium) bicarbonate (NaHCO₃), magnesium carbonate (MgCO₃), and calcium carbonate (CaCO₃), or their combinations are preferred. Stronger bases such as sodium hydroxide, potassium hydroxide or calcium hydroxide can degrade or hydrolyze artesunate during the preparation.

The polyvinyl pyrrolidone (Kollidon® or Povidone) is used as emulsifying polymer to improve the solubility and the stability of artesunate. Furthermore, the emulsion permits to confine artesunate, separated from the environment by a protective emulsifying layer. Such a protective coating can extend shelf life, prevent exposure to gastric acid in stomach and delay the degradation of artesunate.

To obtain a dry powder artesunate from a fluid, several drying processing methods may be used, such as precipitation by using solvents (alcohol or acetone), the freeze-drying or lyophilization. But these processing methods are very long and difficult for industrial manufacturing. In the present application, the spray drying method is preferably used because it is a scalable process and it is widely used to produce dry pharmaceutical powders. Furthermore, spray-drying is rapid, fully automated, continuous, reproducible, single-step, and thus, scalable without major modifications.

### Spray-Drying to Obtain Water Soluble Artesunate Powders

The drying to obtain the water soluble artesunate solid powders was performed using a Pilotech™ YC-510 small spray dryer.

This spray dryer was equipped with a 0.7 mm standard nozzle jet and operated using the following parameters:
Inlet/outlet temperature respectively 150° C. and 90° C.;
Spray flow approximately 500 mL/h;
Airflow setting 40 m³/h;
Peristaltic pump speed 2 or 3 rpm
Blower (fan) setting: 50 Hz
De-blocker frequency 5 seconds
Air pressure 0.44 mPa

The suspension was mixed continuously during the drying process using a magnetic stirrer to ensure homogenous solution.

### Drying and Sieving

Dry the powders obtained by spray drying at an inlet temperature of 30±2° C. in a fluid bed dryer.
Sieve the dried mass through a 600 µm screen to remove large agglomerates that would not dry uniformly.

### Storage

The obtained powders (Water-Soluble Artesunate - WSA) are conserved in a hermetically sealed brown bottle and stored in a dry place at room temperature and relativity humidity ≤45%.

### Characterization

### Solubility

An amount of 0.5 g of different artesunate formulations (untreated artesunate, sodium Artesunate, Artesunate/SG complex and WSA powders) are dispersed in 10 mL of simulated gastric fluid (SGF, pH 1.2). All solutions are incubated at 36.5° C. under mild shaking with (100 rev/min) in a G24 Environmental Incubator Shaker (New Brunswick Scientific Co., NJ, USA). After 30 minutes the solution of untreated Artesunate has a milky appearance, with an important precipitation at the bottom of vial. This is because the Artesunate is mainly under the protonated acid form (—COOH). For artesunate under sodium carbonate salt form, the solution is cloudy, but no evident precipitation is observed. In contrast, the solution of artesunate/SG complex and WSA are soluble, slightly transparent and no precipitations are apparent. Additionally, the solubility of WSA prepared with ratio of artesunate : emulsifying polymer: soluble polymer of 80:10:10 was improved over that of WSA with ratio 90:5:5, because the dispersion of the powder is faster and the solution is clearer.

### FTIR Analysis

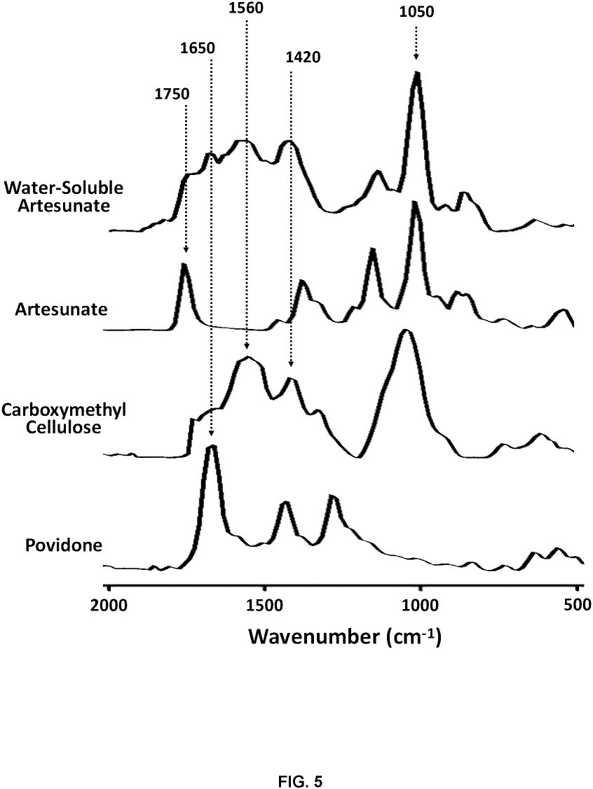

Now referring to the FIG. 5, typically absorption bands for artesunate (under acidic form, —COOH) are located 1750 cm⁻¹, assigned to carboxylic group from succinic residue. No absorption band at 1590 and 1410 cm⁻¹ (assigned for carboxylate form) is detected. For Carboxymethyl cellulose spectrum, absorption band located at 1750 cm⁻¹ is attributed for stretching vibration of carboxylic acid (—COOH) whereas bands at 1550 and 1420 cm⁻¹ are assigned for asymmetric and symmetric stretching vibration of carboxylate (—COO⁻) groups. For Povidone (Polyvinyl pyrrolidone), an absorption band at 1650 cm⁻¹ is assigned for carbonyl group (C═O) and that at 1275 cm⁻¹ attributed for C—N bond.

Regarding Water-Soluble Artesunate complex spectrum, all absorption bands of components are detected: Carboxymethyl cellulose (1750, 1560 and 1420 cm⁻¹); Povidone (1650 and 1275 cm⁻¹); Artesunate (1750 cm cm⁻¹).

### Example 2

### Other Composition for the Preparation of Water-Soluble Artesunate Solid Powder

Water-Soluble Artesunate can be obtained with numerous polymeric compounds. For soluble polymers, it is possible to use Carboxymethyl cellulose, Carboxymethyl starch, Maltodextrin, Cyclodextrin, Dextran, Gellan gum, etc., or combination thereof. For emulsifying polymers, it is possible to use Albumin, Collagen, Gelatin, Povidone, Hydroxypropyl cellulose, (Hydroxypropyl)methyl cellulose, Methyl cellulose, Octenyl succinate starch, Polyvinyl alcohol, Polyvinyl acetate, Polysorbate, etc. or combination thereof.

### Preparation of Artesunate/Xanthan Gum/Octenyl Succinate Starch Complex

In this case, different soluble or emulsifying polymers can be used to produce Water-Soluble Artesunate. Practically, Xanthan Gum (50-100 centipoise, Sigma, USA) and Octenyl Succinate Starch (CLEARGUM® CO 03, Roquette, France) are used.

Water-Soluble Artesunate (ratio Artesunate/Xanthan Gum/Octenyl Succinate Starch, 90:5:5) is prepared in the same preparation conditions as described in the Example 1, above.

### Example 3

### Preparation of Artesunate/Hydroxypropyl Beta-Cyclodextrin/(Hydroxypropyl) Methyl Cellulose Complex

Hydroxypropyl beta-Cyclodextrin (Kleptose® HP Oral Grade, Roquette, France) and (Hydroxypropyl)methyl cellulose (viscosity 50-200 centipoise, Sigma, USA) are used. Water-Soluble Artesunate (ratio Artesunate/Hydroxypropyl beta-cyclodextrin/(Hydroxypropyl)methyl cellulose, 90:5:5) is prepared in the same preparation conditions as described in the Example 1, above.

### Example 4

### Antiviral Activity of Water-Soluble Artesunate Against Sars Cov-2

This study consists in detecting antiviral activity of different preparations of Artesunate: Sodium Artesunate, Artesunate/Starch Glycolate complex and Water-Soluble Artesunate (Artesunate/CMC/Povidone complex, ratio 90:5:5).

### Preparation of Artesunate Compounds

The Water-Soluble Artesunate is diluted with sterile Millipure™ water to a stock working concentration of 25 mg/ml. Further dilutions to experimental concentrations are made with DMEM + 2% FBS.

### Cells

VERO E6 cells are plated into 96-well flat bottom plates at a density 15,000 cells per well. Cells are maintained in DMEM + 10% FBS + Penicillin and Streptomycin up until time of infection or drug treatment. Post infection cells are maintained in DMEM + 2%FBS.

### Virus

SARS-CoV-2 (isolate USA-WA1-2020) at a titer of 1×10^{5.8} IU/ml is used to infect VERO E6 cells. Infection is conducted in serum-free DMEM for 60 minutes with agitation every 15 minutes. Post infection virus is removed, and fresh media is replaced on the cells with or without study compounds.

### Monitoring Virus Replication With Compounds

Virus replication in the presence of compounds is monitored after 48 h and the viral supernatants is harvested, and viral RNA is isolated using Qiagen™ QlAAMP™ Viral RNA mini kits. The quantity of viral RNA was measured by quantitative reverse transcriptase polymerase chain reaction (qPCR) utilizing primers specific to SARS-CoV-2 spike protein.

Cell viability is measured with Water-Soluble Artesunate in the absence of virus utilizing Promega™ CellTiter™-Glo 2.0.

### Results

Good results are obtained for Water-Soluble Artesunate (WSA), as shown in Table 1 below, which shows the cycle threshold (Ct) values for the quantity of viral RNA measure in each sample. Low antiviral activity is observed for each of sodium artesunate and Artesunate/Starch Glycolate (Ct = about 23 for each). For sodium Artesunate, it is probably due to its degradation in inactive form during preparation or incubation whereas for Artesunate/Starch Glycolate, it is possibly due to the hinderance of starch glycolate to expose of Artesunate in contact with cell infected cells. WSA delayed appearance of viral RNA until a Ct of 32, indicative of an antiviral activity.

TABLE 1

Ct values for the quantity of viral RNA measure in each sample

| Tested Coumpound | Concentration (µg/mL) | Cycle Threshold Value |
|---|---|---|
| Sodium Artesunate | 160 | 22.5 |
| Complex Artesunate/CMS | 160 | 23.0 |
| WSA | 160 | 32.0 |
| Positive Control * | - | 14.0 |

* The positive control containing noninfectious material consists of in vitro transcribed RNA. Covid-19 positive control yields a positive result with each assay in the Real-Time rt-PCR)

### Example 5

### Antiviral Activity of Water-Soluble Artesunate With Different Ratio Against Sars Cov-2

As mentioned previously in example 1 (Section Solubility), the solubility of WSA with a ratio of 80:10:10 is superior to that of WSA with a ratio of 90:5:5. For this reason, this study aims to assess whether WSA with ratio 80:10:10 possesses an antiviral activity comparable or superior than WSA with ratio 90:5:5. The experience is carried out under the conditions as described in the Example 4. Results indicated that there is no significant difference between the two ratios.

TABLE 2

Ct values for the quantity of viral RNA measure in each sample

| Tested Ratio | Artesunate Concentration (µg/mL) | Cycle Threshold Value |
|---|---|---|
| WSA 80:10:10 | 80 | 20.2 |
| WSA 90:5:5 | 80 | 20.0 |
| Positive Control * | - | 14.0 |

* The positive control containing noninfectious material consists of in vitro transcribed RNA. Covid-19 positive control is yields a positive result with each assay in the Real-Time RT-PCR)

### Example 6

### Study of Antiviral Activity of Water-Soluble Artesunate As a Dose Response & Combined With Telmisartan Against Sars Cov-2

The experiment is carried out under similar conditions as described in Example 4. For the preparation of Telmisartan, this molecule is sparingly soluble in the aqueous medium. For this reason, Telmisartan is dispersed in HEPES Buffered Saline - EDTA Polysorbate.

### Results

Antiviral activity is observed for the WSA, which increases in efficacy when used at more than 80 µg/mL. No activity antiviral is observed for Telmisartan alone (not shown) and no synergistic or additive effect of Telmisartan is observed when combined with WSA. As expected, these results show that Telmisartan works for the blockade of the ACE-2 receptor in order to prevent the spread of the virus in other organs whereas Artesunate exerts its antiviral and anti-inflammatory activity.

TABLE 3

Cycle Threshold values for the quantity of viral RNA measure in each sample

| Tested Coumpound | Concentration (µg/mL) | Cycle Threshold Value |
|---|---|---|
| WSA | 40 | 26.5 |
| WSA | 80 | 26.3 |
| WSA | 160 | 31.4 |
| WSA + Telmisartan | 40 | 26.0 |
| WSA + Telmisartan | 80 | 25.9 |
| WSA + Telmisartan | 160 | 30.0 |
| Positive Control * | - | 14.1 |

*The positive control containing noninfectious material consists of in vitro transcribed RNA. Covid-19 positive control is yields a positive result with each assay in the Real-Time RT-PCR)

### Example 7

### In Vitro Study of Water-Soluble Artesunate Against Sars Cov-2

This in vitro study consists in confirming the antiviral activity of Water-Soluble Artesunate (Artesunate/CMC/Povidone complex, ratio 90:5:5) and determining the half maximal effective concentration (EC₅₀). Additionally, its toxicity is also assessed in Vero E6 Cells.

### Preparation of Artesunate Compounds

The Water-Soluble Artesunate is diluted with sterile Millipure™ water to a stock working concentration of 25 mg/ml. Further dilutions to experimental concentrations are made with DMEM + 2% FBS.

### Cells

VERO E6 cells are plated into 96-well flat bottom plates at a density 15,000 cells per well. Cells are maintained in DMEM + 10% FBS + Penicillin and Streptomycin up until time of infection or drug treatment. Post infection cells are maintained in DMEM + 2%FBS.

### Virus

SARS-CoV-2 (isolate USA-WA1-2020) at a titer of 1×10^{5.8} IU/ml is used to infect VERO E6 cells. Infection is conducted in serum-free DMEM for 60 minutes with agitation every 15 minutes. Post infection virus is removed, and fresh media is replaced on the cells with or without study compound.

### Monitoring Virus Replication With Compounds

Virus replication in the presence of compounds was monitored at 3- and 6-days post-infection. Viral infection of cells was scored by quantification of viral cytopathic effects (vCPE). At days 3 and 6 viral supernatants were harvested and viral RNA was isolated using Qiagen™ QlAAMP™ Viral RNA mini kits. Viral RNA was subjected to qPCR utilizing primers specific to SARS-CoV-2 spike protein. Cell viability is measured with Water-Soluble Artesunate in the absence of virus utilizing Promega™ CellTiter-Glo™ 2.0.

### Monitoring Cell Viability With Compounds in the Absence of Virus

Cell viability was measured at day 6 utilizing Promega™ Cell-Titer-Glo™ 2.0.

### Results

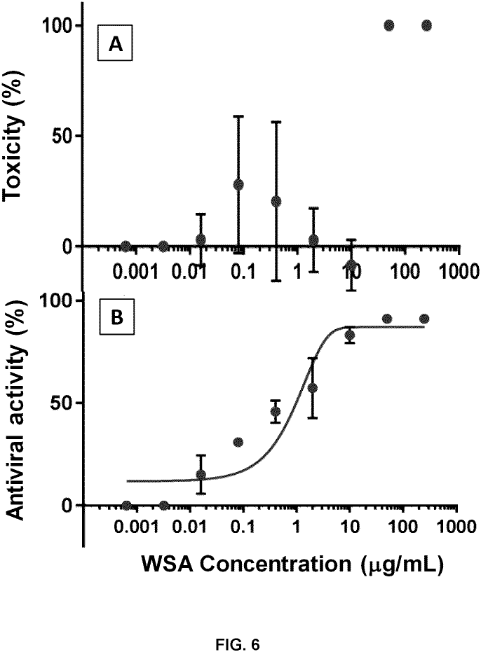

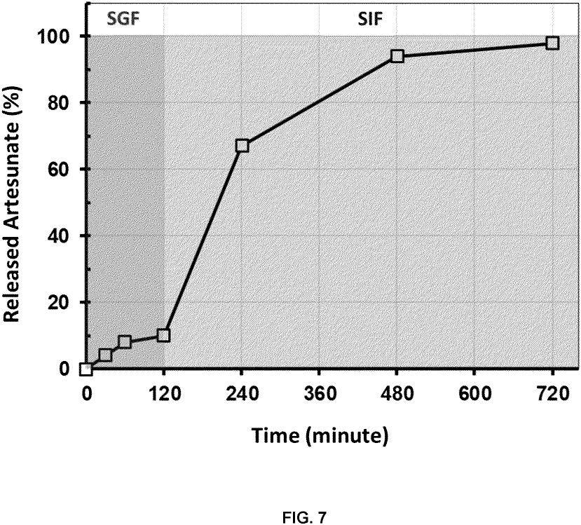

Referring FIG. 6A, the toxicity effect on Vero E6 cells is detected from 50 µg/mL or more of Water-Soluble Artesunate. Indeed, no toxicity is detected at 10 µg/mL, but 100% toxicity is found from 100 µg / mL (FIG. 6A). However, antiviral activity is detected starting at 0.1 µg/mL, and EC₅₀ is 1 µg/mL (FIG. 6B). These effective concentrations are very low (<5 µg / mL) compared with toxicity > 50 µg / mL). The antiviral activity (FIG. 6B) is completely inhibited the virus from 5 µg/mL of Water-Soluble Artesunate.

### Example 8

### Orally Delayed Release Tablet Formulation for Water-Soluble Artesunate Used in Combination With Other Drugs for Treatment of Covid-19

TABLE 4

delayed release formulation

| | Quantity (mg) | Percentage (%) |
|---|---|---|
| Water-Soluble Artesunate powder (containing 60 % Artesunate) | 223 (Corresponding approx. to 200 mg of Artesunate) | 42.08 |
| Eudragit L100-55 | 182 | 34.34 |
| Hydroxypropylmethyl Cellulose | 25 | 4.72 |
| Crospovidone | 90 | 16.98 |
| Magnesium Stearate | 10 | 1.88 |
| TOTAL | 530 | 100.0 |

In the present formulation, Eudragit is used as delayed delivery agent, Hydroxypropyl methylcellulose as stabilizing agent and Crospovidone as disintegrating agent in the intestinal tract.

WSA (ratio 90:5:5) formulated with excipients is directly compacted (2.0 T/cm²) in flatfaced punches (Carver Press, Wabash, IN, USA) to obtain round-shaped tablet.

### In Vitro Dissolution Tests

In vitro release studies of WSA monolithic tablets are carried out using an USP paddle (Apparatus 2) method with a dissolution Distek 5100 apparatus (North Brunswick, NJ, USA) at 100 rpm and 37° C. Now referring to FIG. 6, the artesunate release from monolithic tablets (n = 3) in 1 L of simulated gastric fluid (SGF, pH 1.2) is spectrophotometrically measured at predetermined sampling intervals (0, 0.5, 1.0, 2.0, 4.0, 8.0, 12 and 16 h), volumes of 1 mL were withdrawn from dissolution medium to estimate the concentration of artesunate by spectrophotometry. This method, less sensitive and precise than HPLC, is chosen because it is simple and rapid. Since the maximal absorption of Eudragit is about of 265 nm, the reading for artesunate is selected at 222 nm. The release of artesunate is estimated and expressed as the relative percentage from the total loading.

The kinetic release profiles of Artesunate shows that there is a fast release of about 5-10% of artesunate (corresponding approximately to the 9-18 mg of Artesunate), after 2 h in simulated gastric fluid (SGF) at 37° C. A rapid release is followed for remaining Artesunate which is achieved after 6 h in SIF.

### Example 9

### Carbonate Salts Combination

It is possible to improve solubility of native Artesunate (Artesunic acid) by incorporating carbonate salts, such as sodium carbonate (Na₂CO₃, soda ash) and sodium bicarbonate (NaHCO₃, baking soda) in the tablet formulation. Sodium carbonate has a lower percentage of CO₂ than sodium bicarbonate and can be used for delayed release formulation. It is of interest to mention that potassium (K₂CO₃, KHCO₃), magnesium carbonate (MgCO₃), and calcium carbonate (CaCO₃) salts can also be used.

TABLE 4

Artesunate and carbonate formulation

| | Quantity (mg) | Percentage (%) |
|---|---|---|
| Native Artesunate powder (Artesunic acid) | 200 | 40.0 |
| Sodium Carbonate | 10 | 2.0 |
| Eudragit L100-55 | 180 | 36.0 |
| Crospovidone | 100 | 20.0 |
| Magnesium Stearate | 10 | 2.0 |
| TOTAL | 500 | 100.0 |

The ratio of native artesunate and sodium carbonate may be from 6.0 : 4.0 to 9.9 : 0.1 w/w, and is preferably 9.5:0.5 w/w as illustrated above.

The kinetic release analysis is carried out under similar conditions as described in the example 8.

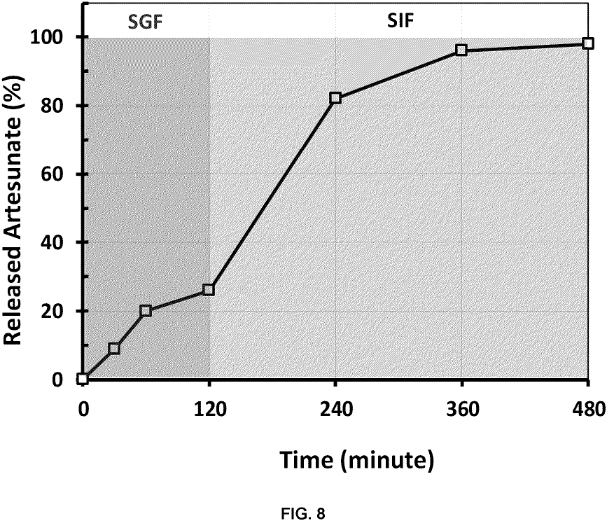

Upon hydration of the tablet, sodium carbonate is first solubilized and deprotonates succinic acid moieties from Artesunic acid to succinate ion, which improve its solubility. The kinetic release of (native) Artesunate is about of 25 % after 2 h in SGF (pH 1.2) and completely release in SIF (pH 6.8) after 5 h (FIG. 8).

Sodium carbonate improves products stability compared to products that contain only sodium bicarbonate. Nevertheless, efficient formulation can be prepared with sodium bicarbonate under effervescent formula.

TABLE 5

Artesunate and bicarbonate formulation

| | Quantity (mg) | Percentage (%) |
|---|---|---|
| Native Artesunate powder (Artesunic acid) | 200 | 37.8 |
| Sodium bicarbonate | 15 | 2.8 |
| Eudragit L100-55 | 140 | 26.4 |
| Crospovidone | 160 | 30.2 |
| Magnesium Stearate | 15 | 2.8 |
| TOTAL | 530 | 100.0 |

### Example 10

### Combination of Water-Soluble Artesunate and Telmisartan for Oral Administration

The Water-Soluble Artesunate can combine with different drugs to treat SARS-CoV-2. In this example, Water-Soluble Artesunate is combined with Telmisartan. In fact, Water-Soluble Artesunate is used for its antiviral activity and its anti-inflammatory whereas Telmisartan is an ACE-2 blocker which is thought to prevent SARS-CoV-2 to penetrate in cells or to spread from lung to another organs.

In this case, it is possible to administrate Water-Soluble Artesunate tablet (formulated under as described previously in the Example-8) with Telmisartan tablet. The dose recommended for this combination Water-Soluble Artesunate/Telmisartan to the patient is once a day (180 mg / 80 mg) or twice a day (180 × 2 tablets / 80 mg × 2 tablets) for 7 days.

### Example 11

### Combination With Other Drugs for Oral Administration

Similarly, the combination of Water-Soluble Artesunate with other drugs is possible such as:
Dexamethasone (an anti-inflammatory)
   Water-Soluble Artesunate / Dexamethasone, once a day (180 mg / 10 mg) or twice a day (180 × 2 tablets / 10 mg × 2 tablets).
Camostat (a serine protease inhibitor):
   Water-Soluble Artesunate / Camostat, once a day (180 mg / 600 mg) or twice a day (180 x× 2 tablets / 600 mg × 2 tablets).
Acetaminophen (an analgesic and antipyretic):
   For Water-Soluble Artesunate tablet, the recommended dose is once a day (180 mg) or twice a day (180 × 2 tablets) whereas the recommended dose for acetaminophen (500 mg) is every 4-6 h, but not to exceed 3000 mg/day.
Umifenovir (an antiviral)
   For Water-Soluble Artesunate / Umifenovir (Arbidol), the recommended dose is once a day (180 mg / 100 mg) or twice a day (180 mg x 2 tablets / 100 mg x 2 tablets).
Fluvoxamine (a selective serotonin reuptake inhibitor and σ-1 receptor agonist)
   For Water-Soluble Artesunate / Fluvoxamine, the recommended dose is once a day (180 mg / 100 mg) or twice a day (180 mg x 2 tablets / 100 mg x 2 tablets).
Cholecalciferol (an immunomodulatory and anti-inflammatory)
   For Water-Soluble Artesunate tablet, the recommended dose is once a day (180 mg) or twice a day (180 × 2 tablets) whereas the recommended dose for Cholecalciferol is 4000 IU once per day.

### Example 12

### Combination Formulation of Water-Soluble Artesunate With Other Drugs Under Monolithic Tablet Dosage Form

It is possible to combine Water-Soluble Artesunate with another drug in one tablet. For example, Water-Soluble Artesunate can mix with zinc gluconate and formulate under delayed release monolithic tablet dosage form Table 5.

The recommended dose is once per day (180 mg / 60 mg) or twice per day (180 mg × 2 tablets / 60 mg × 2 tablets).

TABLE 6

Delayed release used for combination formulation

| | Quantity (mg) | Percentage (%) |
|---|---|---|
| Water-Soluble Artesunate powder (containing 60 % Artesunate) | 223 (corresponding approx. to 200 mg of Artesunate) | 33.04 |
| Zinc gluconate | 60 | 8.89 |
| Eudragit L100-55 | 222 | 32.89 |
| Hydroxypropylmethyl Cellulose | 40 | 5.92 |
| Crospovidone | 120 | 17.78 |
| Magnesium Stearate | 10 | 1.48 |
| TOTAL | 675 | 100.0 |

### Example 13

### Combination Formulation of Water-Soluble Artesunate With Different Dosage Forms

Water-Soluble Artesunate is preferable formulated under tablet dosage form for oral administration. When combined with another drug, the latter could be administrated by intravenous, perfusion or inhalation, etc.

By example, it is possible to combine Water-Soluble Artesunate with Anakinra (an IL-1 receptor antagonist). In this case, Water-Soluble Artesunate tablet is orally administrated whereas Anakinra is administrated by intravenous (50-100 mg every 6 hours)

While preferred embodiments have been described above and illustrated in the accompanying drawings, it will be evident to those skilled in the art that modifications may be made without departing from this disclosure. Such modifications are considered as possible variants comprised in the scope of the disclosure.

1. A method of treating or preventing a viral infection in a subject in need thereof comprising administering a therapeutically effective amount of artesunate or pharmaceutically acceptable salts thereof, and stereoisomers thereof to said subject.

2. The method of claim 1, wherein said artesunate is provided as a therapeutically effective amount of a delayed release dosage form comprising:
an artesunate or pharmaceutically acceptable salts thereof, in combination with a carbonate salt;
an artesunate emulsion having a pH value of from about 7.5 to 8.0 and comprising an artesunate or pharmaceutically acceptable salts thereof, and stereoisomers thereof stabilized with an emulsifying polymer and a soluble polymer,
or a combination thereof,
to said subject.

3. The method of claim 2, wherein said artesunate emulsion comprises from about 25% to about 90% w/w of said artesunate pharmaceutically acceptable salt.

4. The method of claim 2, wherein said artesunate emulsion comprises from about 80% to about 90% w/w of said artesunate pharmaceutically acceptable salt.

5. The method of claim 2, wherein said artesunate emulsion comprises from about 1% to about 55% w/w or from about 5% to about 10% w/w of said emulsifying polymer.

6. (canceled)

7. The method of claim 2, wherein said artesunate emulsion comprises from about 1% to about 55% w/w, or from about 5% to about 10% w/w of said soluble polymer.

8. (canceled)

9. The method of claim 2, wherein said emulsifying polymer is a nonionic, an anionic, a cationic, an amphoteric polymer, or a combination thereof.

10. The method of claim 2, wherein said emulsifying polymer is selected from the group consisting of a (hydroxypropyl)methyl cellulose, a methyl cellulose, an ethyl cellulose, an acetyl cellulose, an octenyl succinate starch, a hydroxypropyl starch, a polyvinyl pyrrolidone, a polyvinyl acetate-acrylate, a poloxamer, an albumin, a gelatin or combinations thereof.

11. The method of claim 10, wherein said emulsifying polymer is polyvinyl pyrrolidone.

12. The method of claim 2, wherein said soluble polymer is a nonionic, an anionic, a cationic, an amphoteric polymer, or a combination thereof.

13. The method of claim 2, wherein said soluble polymer is selected from the group consisting of a carboxymethyl cellulose, a carboxymethyl starch, a carboxyethyl starch, a succinyl starch, a distarch glycerol, a distarch phosphate, a hydroxypropyl distarch glycerol, a hydroxypropyl distarch phosphate, a maltodextrin, a cyclodextrin, an acacia gum, a pectin, an amylopectin, a carrageenan, a xanthan gum, a tragacanth gum, a guar gum or combination thereof.

14. The method of claim 13, wherein said soluble polymer is carboxymethyl cellulose.

15. The method of claim 2, wherein said pH value is obtained with a weak base.

16. The method of claim 15, wherein said weak base is a carbonate salt.

17. The method of claim 2, wherein said carbonate salt is selected from the group consisting of sodium carbonate (Na₂CO₃), potassium carbonate (K₂CO₃), sodium bicarbonate (NaHCO₃), and potassium bicarbonate (KHCO₃), magnesium carbonate (MgCO₃), and calcium carbonate (CaCO₃), and combinations thereof.

18. The method of claim 17, wherein said carbonate salt is from about 1% to about 40% w/w, or about 5% w/w said delayed release dosage form or said artesunate emulsion.

19. (canceled)

20. The method of claim 1, further comprising administering a second therapeutic agent.

21. The method of claim 20, wherein said second therapeutic agent is an antiviral agent, an anti-inflammatory drug, an immunomodulator, an Angiotensin Converting Enzyme (ACE) inhibitor, a chemotherapeutic agent, a protease inhibitor, fluxamine, and combinations thereof.

22. The method of claim 21, wherein said antiviral agent is Favipiravir, Abacavir, Acyclovir, Adefovir, Amantadine, Ampligen, Amprenavir, Arbidol, Atazanavir, Atripla, Balavir, Baloxavir marboxil, Biktarvy, Boceprevir, Cidofovir, Cobicistat, Combivir, Daclatasvir, Darunavir, Delavirdine, Descovy, Didanosine, Docosanol, Dolutegravir, Doravirine, Ecoliever, Edoxudine, Efavirenz, Elvitegravir, Emtricitabine, Enfuvirtide, Entecavir, Etravirine, Famciclovir, Fomivirsen, Fosamprenavir, Foscarnet, Fosfonet, Ganciclovir, Ibacitabine, Ibalizumab, Idoxuridine, Imiquimod, Imunovir, Indinavir, Inosine, an Integrase inhibitor, Interferon type I, Interferon type II, Interferon type III, Interferon, Lamivudine, Letermovir, Lopinavir, Loviride, Maraviroc, Methisazone, Moroxydine, Nelfinavir, Nevirapine, Nexavir, Nitazoxanide, Norvir, a nucleoside analogues, Oseltamivir, Peginterferon alfa-2a, Peginterferon alfa-2b, Penciclovir, Peramivir, Pleconaril, Podophyllotoxin, a protease inhibitor, Pyramidine, Raltegravir, Remdesivir, a reverse transcriptase inhibitor, Ribavirin, Rilpivirine, Rimantadine, Ritonavir, Saquinavir, Simeprevir, Sofosbuvir, Stavudine, Telaprevir, Telbivudine, Tenofovir alafenamide, Tenofovir disoproxil, Tenofovir, Tipranavir, Trifluridine, Trizivir, Tromantadine, Truvada, Umifenovir, Valaciclovir, Valganciclovir, Vicriviroc, Vidarabine, Viramidine, Zalcitabine, Zanamivir, Zidovudine, Tilorone, Mepacrine, pyronaridine and combinations thereof;
wherein said anti-inflammatory drug is a Non-Steriodal Anti-Inflammatory Drugs (NSAIDs) comprising aspirin, celecoxib, diclofenac, etodolac, ibuprofen, indomethacin, ketoprofen, ketorolac, nabumetone, naproxen, oxaprozin, piroxicam, salsalate, sulindac, tolmetin, indomethacin, including colchicine, and analgesic such as acetaminophane and combinations thereof
wherein said anti-inflammatory drug is a corticosteroidal drug comprising Dexamethasone, Betamethasone, Prednisone, Prednisolone, Methylprednisolone. Triamcinolone, Budesonide, Flunisolide and combinations thereof:
wherein said immunomodulator is Anakinra, Canakinumab, Tocilizumab, Sarilumab, Baricitinib, Fedratinib, Ruxolitinib, Fingolimob, Infliximab, Adalimumab, and combinations thereof,
wherein said ACE inhibitor or ACE blocker is Benazepril, captopril, Enalapril, Fosinopril, Lisinopril, Moexipril, Perindopril, Quinapril, Ramipril, Trandolapril, telmisartan, candesartan, Irbesartan, Valsartan, Losarian, Olmesartan, Eprosartan, Azilsartan and combinations thereof;
wherein said chemotherapeutic agent is Daunorubicin, Mitoxantrone, Metamizole and combinations thereof; and
wherein said protease inhibitor is Camostal, Nafamostat, Gabexate and combinations thereof.

23-28. (canceled)

29. The method of claim 1, wherein said viral infection is a corona virus infection or a SARS-CoV-2 infection.

30-65. (canceled)
